(19) United States
(12) Patent Application Publication
(10) Pub. No.: US 2018/0363050 A1
(43) Pub. Date: Dec. 20, 2018

(54) MULTIPLEXING IN PARTITIONS USING PRIMER PARTICLES

(71) Applicant: Raindance Technologies, Inc., Billerica, MA (US)

(72) Inventors: Brian Hutchison, Medford, MA (US); Darren R. Link, Lexington, MA (US); Zuwei Ma, Billerica, MA (US); Qun Zhong, Lexington, MA (US); Aisling Steele, Billerica, MA (US)

(21) Appl. No.: 15/782,001

(22) PCT Filed: Dec. 7, 2016
(86) PCT No.: PCT/US16/65430

§ 371 Date: Jun. 6, 2018

Related U.S. Application Data
(60) Provisional application No. 62/264,187, filed on Dec. 7, 2015.

(51) Int. Cl. C12Q 1/6876; C12Q 1/6874

(52) U.S. Cl. CPC C12Q 1/6876; C12Q 1/6874

(57) ABSTRACT

Described herein are microparticles each comprising a plurality of bound biological molecules. Further described herein is a plurality of microdroplets each comprising one or more primer vehicles. Methods of making and using these microdroplets are also reported. An exemplary microparticle is of Formula (I).

### CROSS-REFERENCE TO RELATED APPLICATION

This application claims the benefit of and priority to U.S. Provisional Application Serial No. 62/264,187, filed December 7, 2015, the content of which is incorporated by reference herein in its entirety.

### FIELD OF THE INVENTION

The invention generally relates to microdroplets comprising one or more primer vehicles and methods of use thereof.

### BACKGROUND

Microfluidic technologies for generating droplets into an immiscible fluid have been developed and used for many purposes that include performing various biochemical reactions in a massively parallel format. The microfluidic technologies provided a significant advancement over previously used bulk droplet generation methods that include performing PCR reactions in the droplets in a multiplex format where multiple primer species each directed to a different target region are present in the droplets. One difficulty, however, is how to control the distribution of the primer species and sample in the droplets in a way that can maximize the usage of available droplets.

For instance, some embodiments have resorted to using approaches that pre-combine sample and soluble reagents into an aqueous solution used to form droplets in an immiscible fluid. These approaches do not provide control over the distribution of reagents into the droplets other than to control the original concentration in the aqueous solution. Alternatively, some embodiments employ strategies that provide control of reagent distribution by merging a first droplet (e.g. containing the primer species) with a second droplet and/or stream of fluid (e.g. containing sample) in order to control the delivery of reagents into the droplets, but such approaches require complicated and expensive microfluidic platforms.

Therefore, simplified, cost effective, and adaptable approaches to efficiently generate microdroplets with a desired distribution of reagents are highly desirable.

### SUMMARY

Embodiments of the invention relate to the fields of nucleic acid amplification and sequencing. More particularly, embodiments of the invention relate to microdroplets comprising one or more primer vehicles.

In one aspect, provided herein is a microparticle of Formula (I):

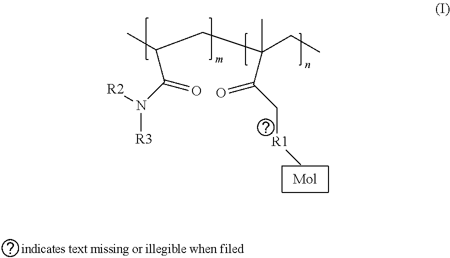

wherein [symbol] is a biological molecule;
m is an integer of 0 to 100, inclusive;
n is an integer of 0 to 100, inclusive;
R1 is a binding moiety selected from the group consisting of a bond, optionally substituted alkylene, optionally substituted heteroalkylene, optionally substituted alkenylene, optionally substituted heteroalkenylene, optionally substituted alkynylene, optionally substituted heteroalkynylene, optionally substituted heterocyclylene, optionally substituted heteroarylene, and
each of R2 and R3 is independently hydrogen, substituted or unsubstituted alkyl, or a nitrogen protecting group.

In certain embodiments, the microparticle of Formula (I) is of Formula (II):

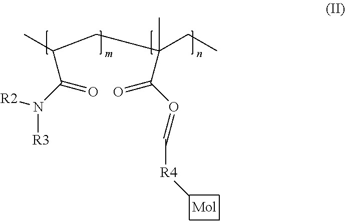

wherein R4 is optionally substituted alkylene, optionally substituted alkenylene, optionally substituted alkynylene, optionally substituted heterocyclylene, or optionally substituted heteroarylene.

In certain embodiments, the microparticle of Formula (I) is of Formula (II-a):

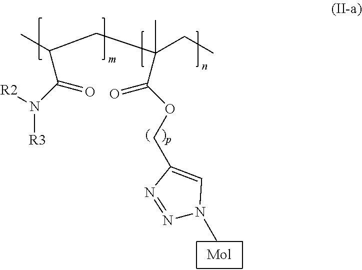

wherein p is an integer of 1 to 5, inclusive.

In certain embodiments, the microparticle of Formula (I) is of Formula (II-b):

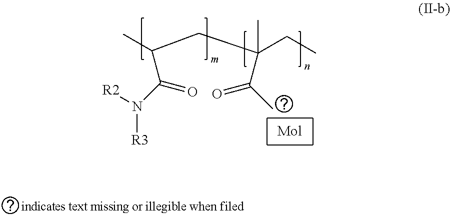

In certain embodiments, provided herein is a method of preparing the microparticle of Formula (I) comprising contacting a compound of Formula (i):

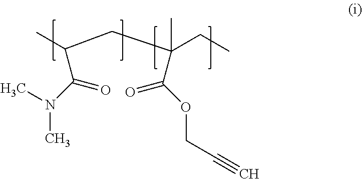

with at least one compound of Formula (ii):

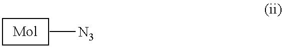

or a salt thereof.

In certain embodiments, the method of preparing the microparticle of Formula (I) further comprises contacting a compound of Formula (iii):

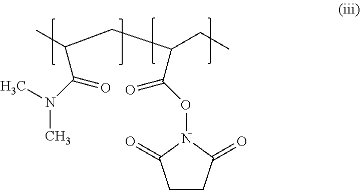

with at least one compound of Formula (iv):

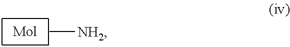

or a salt thereof.

In another aspect, provided herein is a microparticle comprising a plurality of biological molecules, wherein each biological molecule is bound to the microparticle through a binding moiety. The interaction between the biological molecule and the binding moiety can be one or more covalent bonds or hydrogen bonds. In certain embodiments, the binding moiety is formed by a Click Reaction. In certain embodiments, the binding moiety comprises a triazole moiety.

In certain embodiments, the biological molecule is a nucleic acid. In certain embodiments, the biological molecule is DNA or RNA. In certain embodiments, the biological molecule is an oligonucleotide sequence of about 3 to about 30 bases in length. In certain embodiments, the biological molecule is an oligonucleotide sequence of about 15 to about 25 bases in length. In certain embodiments, the biological molecule is a primer member. In certain embodiments, the biological molecule is a DNA sequence of about 15 to about 25 bases in length.

In another aspect, provided herein is a microdroplet library comprising a plurality of microdroplets, each comprising a nucleic acid template molecule and a plurality of primer vehicles, wherein each primer vehicle comprises a plurality of primer species bound to a microparticle through a plurality of binding moieties, wherein each primer species is specific for a different target site of a nucleic acid template molecule.

In certain embodiments, the primer species is a primer pair. In certain embodiments, the primer species is a member of a primer pair. In certain embodiments, the primer species is a single oligonucleotide. In certain embodiments, the single oligonucleotide further comprises a barcode. In certain embodiments, the barcode is unique to each microdroplet and different between microdroplets. In certain embodiments, the primer species comprises a barcode and a random hexamer. In certain embodiments, the primer species comprises a barcode and a universal sequence. In certain embodiments, the primer species comprises a barcode, a universal sequence, and a target specific sequence. In certain embodiments, the primer species is tri-partite, with a universal tail portion (e.g., oligonucleotide sequences for use in sequencing library construction) immediately 5′ to a barcode sequence, followed by one of a set of random hexamer bases that enable priming from multiple places in the genome.

In another aspect, provided herein is a primer vehicle library comprising a plurality of primer vehicles as described herein. Further provided herein is a microdroplet library comprising a plurality of microdroplets, each comprising a nucleic acid template molecule and a plurality of primer vehicles, wherein each primer vehicle comprises a plurality of primer pairs bound to a microparticle through a plurality of binding moieties, wherein each primer pair is specific for a nucleic acid template molecule and comprises two members each specific for a different target site on the nucleic acid template molecule.

In certain embodiments, at least one microdroplet comprises two or more nucleic acid template molecules. In certain embodiments, at least one microdroplet comprises a single nucleic acid template molecule. In certain embodiments, at least one primer species is specific for a target site on the nucleic acid template molecule in at least one microdroplet. In certain embodiments, at least one member of the primer pairs is specific for a target site on the nucleic acid template molecule in at least one microdroplet. In certain embodiments, at least one primer species is specific to the nucleic acid template in each microdroplet. In certain embodiments, at least one primer species is specific to the nucleic acid template in each microdroplet. In certain embodiments, at least one primer pair is specific to the nucleic acid template in each microdroplet. In certain embodiments, at least two primer pairs each are specific to the nucleic acid template in each microdroplet. In certain embodiments, at least two primer species each are specific to the nucleic acid template in each microdroplet.

The microparticles each can be functionalized with at least one binding moiety. The binding moiety can either form one or more bonds with a primer species. In certain embodiments, the primer species can be ligated to the microparticle through the binding moiety. In certain embodiments, primer species can hybridize with the binding moiety by forming, for example, hydrogen bonds. In certain embodiments, the binding moieties in a microdroplet are the same. In certain embodiments, at least one binding moiety in a microdroplet is different. In certain embodiments, the binding moiety comprises a sequence complementary to a primer species. In certain embodiments, the binding moiety comprises a poly-alanine sequence.

In certain embodiments, each microdroplet contains up to about 200 primer vehicles. In certain embodiments, each microdroplet contains up to about 100 primer vehicles. In certain embodiments, each microdroplet contains up to about 90 primer vehicles. In certain embodiments, each microdroplet contains up to about 80 primer vehicles. In certain embodiments, each microdroplet contains up to about 70 primer vehicles. In certain embodiments, each microdroplet contains up to about 60 primer vehicles. In certain embodiments, each microdroplet contains up to about 50 primer vehicles. In certain embodiments, each microdroplet contains about 10 to about 50 primer vehicles. In certain embodiments, each microdroplet contains about 10 to about 30 primer vehicles. In certain embodiments, each microdroplet contains about 25 primer vehicles. In certain embodiments, each microdroplet contains about 5 to about 10 primer vehicles.

The primer vehicle is a complex comprising a plurality of primer species bound to a microparticle through a plurality of binding moieties. In certain embodiments, the primer vehicle is a complex comprising a plurality of primer pairs bound to a microparticle through a plurality of binding moieties. In certain embodiments, the primer vehicle comprises at least one primer species. In certain embodiments, the primer vehicle comprises at least one primer pair. In certain embodiments, the primer vehicle has a single primer species bound. In certain embodiments, the primer vehicle has a single primer pair bound. In certain embodiments, the primer vehicle has a single oligonucleotide bound. In certain embodiments, the primer vehicle has multiple copies of a single primer species bound. In certain embodiments, the primer vehicle has different primer species bound. In certain embodiments, the primer vehicle has different primer species bound. In certain embodiments, the primer vehicle has different primer pairs bound. In certain embodiments, the primer vehicle has at least two different primer species bound. In certain embodiments, the primer vehicle has at least three different primer species bound. In certain embodiments, the primer vehicle has at least four different primer species bound. In certain embodiments, the primer vehicle has at least five different primer species bound. In certain embodiments, the primer vehicle has at least two different primer pairs bound. In certain embodiments, the primer vehicle has at least three different primer pairs bound. In certain embodiments, the primer vehicle has at least four different primer pairs bound. In certain embodiments, the primer vehicle has at least five different primer pairs bound.

In certain embodiments, each primer vehicle in a microdroplet has a single primer species bound. In certain embodiments, each primer vehicle in a microdroplet has a single primer pair bound. In certain embodiments, each primer vehicle in a microdroplet has multiple copies of a single primer species bound. In certain embodiments, each primer vehicle in a microdroplet has multiple copies of a single primer pair bound. In certain embodiments, each primer vehicle in a microdroplet has different primer species bound. In certain embodiments, each primer vehicle in a microdroplet has different primer pairs bound. In certain embodiments, each primer vehicle in a microdroplet has at least two different primer species bound. In certain embodiments, each primer vehicle in a microdroplet has at least two different primer pairs bound. In certain embodiments, each primer vehicle in a microdroplet has at least three different primer species bound. In certain embodiments, each primer vehicle in a microdroplet has at least three different primer pairs bound. In certain embodiments, each primer vehicle in a microdroplet has at least four different primer species bound. In certain embodiments, each primer vehicle in a microdroplet has at least four different primer pairs bound. In certain embodiments, each primer vehicle in a microdroplet has at least five different primer species bound. In certain embodiments, each primer vehicle in a microdroplet has at least five different primer pairs bound.

In certain embodiments, each microdroplet has a plurality of same primer vehicles. “Same primer vehicles” means the same microparticles each having the same primer species bound. In certain embodiments, each microdroplet has a plurality of same primer vehicles each having the same single primer species bound. In certain embodiments, each microdroplet has a plurality of same primer vehicles, wherein each primer vehicle comprises different primer species bound.

In certain embodiments, each microdroplet has a plurality of different primer vehicles. “Different primer vehicles” means the either microparticle is different between the primer vehicles, or one or more bound primer species are different between the primer vehicles. In certain embodiments, each microdroplet has a plurality of different primer vehicles, wherein each primer vehicle comprises different primer species bound.

In certain embodiments, at least one microdroplet of the plurality of microdroplets has at least one different primer vehicle between the microdroplets. In certain embodiments, the different primer vehicle between the microdroplets comprises a different single primer species bound. In certain embodiments, the different primer vehicle between the microdroplets comprises different primer species bound.

In certain embodiments, the primer species are released from the primer vehicles upon a triggering event. For example, the interaction between the binding moiety and the primer species can break completely or partially upon a triggering event. Exemplified triggers include, but are not limited to chemical triggers (e.g. pH trigger), biological triggers (e.g. enzymatic triggers), thermal triggers, electrical triggers, illuminating triggers, and/or magnetic triggers. In certain embodiments, the trigger is elevated temperature, UV, and/or ultrasound. In certain embodiments, the trigger is elevated temperature. In certain embodiments, the elevated temperature is lower than the denature temperature of a polymerase chain reaction (PCR). In certain embodiments, the elevated temperature is lower than about 90° C. In certain embodiments, the elevated temperature is lower than about 85° C. In certain embodiments, the elevated temperature is lower than about 80° C.

In certain embodiments, the plurality of microdroplets further comprises a plurality of probes, wherein each probe hybridizes to a specific region in one of the target sites. In certain embodiments, the single nucleic acid template is a DNA or an RNA molecule. In certain embodiments, the plurality of microdroplets further comprises reagents for conducting an amplification reaction, i.e. polymerase chain reaction (PCR). In certain embodiments, the probe contains a detectable label. In certain embodiments, at least one probe comprises a different detectable label. In certain embodiments, the microparticle is a bead. The bead can further comprise a polymer. In certain embodiments, the bead comprises self-assembled-DNA nanoparticles. In certain embodiments, the bead is paramagnetic or super-paramagnetic. In certain embodiments, the bead has a functionalized surface. In certain embodiments the bead is functionalized to comprise a binding moiety. In certain embodiments, the binding moiety is streptavidin. In certain embodiments the bead has a silica shell. In certain embodiments, the bead is about 1 to about 1000 nanometers in diameter. In certain embodiments, the bead is about 1 to about 500 nanometers in diameter. In certain embodiments, the bead is about 1 to about 100 nanometers in diameter. In certain embodiments the bead is about 1 to about 90 micron in diameter. In certain embodiments the bead is about 1 to about 80 micron in diameter. In certain embodiments the bead is about 1 to about 70 micron in diameter. In certain embodiments the bead is about 1 to about 60 micron in diameter. In certain embodiments the bead is about 1 to about 50 micron in diameter. In certain embodiments the bead is about 1 to about 40 micron in diameter. In certain embodiments the bead is about 1 to about 30 micron in diameter. In certain embodiments the bead is about 1 to about 20 micron in diameter. In certain embodiments the bead is about 1 to about 10 micron in diameter.

It is understood that when a droplet comprises a single nucleic acid template, that droplet may contain more than one molecules of nucleic acid.

In certain embodiments, the nucleic acid template molecule is a DNA or an RNA.

In certain embodiments, the plurality of microdroplets as described herein each further comprises reagents for conducting a polymerase chain reaction. In certain embodiments, each microdroplet further comprises a probe. In certain embodiments, the probe comprises a detectable label.

The plurality of microdroplets as described herein may be surrounded by an immiscible carrier. In certain embodiments, the immiscible carrier is an oil. In certain embodiments, the immiscible carrier is a fluorocarbon oil (e.g. perfluorocarbon oil).

In certain embodiments, the microparticle has a loading capacity of about from about 10² to about 10¹⁰ members of primer species. In certain embodiments, the microparticle has a loading capacity of about from about 10² to about 10⁹ members of primer species. In certain embodiments, the microparticle has a loading capacity of about from about 10² to about 10⁸ members of primer species. In certain embodiments, the microparticle has a loading capacity of about from about 10² to about 10⁷ members of primer species. In certain embodiments, the microparticle has a loading capacity of about from about 10² to about 10⁶ members of primer species. In certain embodiments, the microparticle has a loading capacity of about from about 10² to about 10⁵ members of primer species. In certain embodiments, the microparticle has a loading capacity of about from about 10² to about 10⁴ members of primer species. In certain embodiments, the microparticle has a loading capacity of about from about 10² to about 10³ members of primer species. In certain embodiments, the microparticle has a loading capacity of about from about 10³ to about 10⁹ members of primer species. In certain embodiments, the microparticle has a loading capacity of about from about 10⁴ to about 10⁸ members of primer species. In certain embodiments, the microparticle has a loading capacity of about from about 10⁵ to about 10⁷ members of primer species. In certain embodiments, the microparticle is a bead with at least 1.0 million bound primer species. In certain embodiments, the microparticle is a bead with at least 10 million bound primer species.

The provided primer vehicle library can be stable for storage. In certain embodiments, the provided primer vehicle library is stable at room temperature for over 3 days. In certain embodiments, the provided primer vehicle library is stable at room temperature for over a week. In certain embodiments, the provided primer vehicle library is stable at room temperature for over two weeks. In certain embodiments, the provided primer vehicle library is stable at room temperature for over three weeks. In certain embodiments, the provided primer vehicle library is stable at room temperature for over four weeks. In certain embodiments, the provided primer vehicle library is stable at room temperature for over two months. In certain embodiments, the provided primer vehicle library is stable at room temperature for over three months. In certain embodiments, the provided primer vehicle library is stable at room temperature for over 3 days. In certain embodiments, the provided primer vehicle library is stable at 4° C. for over a week. In certain embodiments, the provided primer vehicle library is stable at 4° C. for over two weeks. In certain embodiments, the provided primer vehicle library is stable at 4° C. for over three weeks. In certain embodiments, the provided primer vehicle library is stable at 4° C. for over four weeks. In certain embodiments, the provided primer vehicle library is stable at 4° C. for over two months. In certain embodiments, the provided primer vehicle library is stable at 4° C. for over three months. In certain embodiments, the provided primer vehicle library is stable below 0° C. for over a week. In certain embodiments, the provided primer vehicle library is stable at 0° C. for over two weeks. In certain embodiments, the provided primer vehicle library is stable at 0° C. for over three weeks. In certain embodiments, the provided primer vehicle library is stable at 0° C. for over four weeks. In certain embodiments, the provided primer vehicle library is stable at 0° C. for over two months. In certain embodiments, the provided primer vehicle library is stable at 0° C. for over three months. In certain embodiments, the provided primer vehicle library is stable at 0° C. for over one year. In certain embodiments, the provided primer vehicle library is stable at 0° C. for over three years.

The provided microdroplet library can be stable for storage. In certain embodiments, the provided microdroplet library is stable at room temperature for over 3 days. In certain embodiments, the provided microdroplet library is stable at room temperature for over a week. In certain embodiments, the provided microdroplet library is stable at room temperature for over two weeks. In certain embodiments, the provided microdroplet library is stable at room temperature for over three weeks. In certain embodiments, the provided microdroplet library is stable at room temperature for over four weeks. In certain embodiments, the provided microdroplet library is stable at room temperature for over two months. In certain embodiments, the provided microdroplet library is stable at room temperature for over three months. In certain embodiments, the provided microdroplet library is stable at room temperature for over 3 days. In certain embodiments, the provided microdroplet library is stable at 4° C. for over a week. In certain embodiments, the provided microdroplet library is stable at 4° C. for over two weeks. In certain embodiments, the provided microdroplet library is stable at 4° C. for over three weeks. In certain embodiments, the provided microdroplet library is stable at 4° C. for over four weeks. In certain embodiments, the provided microdroplet library is stable at 4° C. for over two months. In certain embodiments, the provided microdroplet library is stable at 4° C. for over three months. In certain embodiments, the provided microdroplet library is stable below 0° C. for over a week. In certain embodiments, the provided microdroplet library is stable at 0° C. for over two weeks. In certain embodiments, the provided microdroplet library is stable at 0° C. for over three weeks. In certain embodiments, the provided microdroplet library is stable at 0° C. for over four weeks. In certain embodiments, the provided microdroplet library is stable at 0° C. for over two months. In certain embodiments, the provided microdroplet library is stable at 0° C. for over three months. In certain embodiments, the provided microdroplet library is stable at 0° C. for over one year. In certain embodiments, the provided microdroplet library is stable at 0° C. for over three years.

In another aspect, provided herein is a method of detecting a nucleic acid template molecule in a biological sample, comprising the steps of:
a) forming a plurality of microdroplets of any one of the preceding claims;
b) amplifying at least one nucleic acid template molecule in the microdroplets to give an amplified product; and
c) sequencing the amplified product.

As used herein, amplification refers to replicating a portion or the entire sequence of the nucleic acid template. In certain embodiments, the replication can be DNA from DNA or DNA from RNA (cDNA). There can be a single replication of the nucleic acid template, there can be a linear amplification of the nucleic acid template or an exponential amplification of the nucleic acid template such as Polymerase Chain Reaction (PCR) or multi-strand displacement amplification. The reagents for conducting the amplification can include such things as polymerase, reverse transcriptase, nucleotides, buffers, etc.). In certain embodiments, the amplification is a linear extension and the primer vehicle further comprises a barcode. In certain embodiments, the primer member on the primer vehicle further comprises a barcode. The barcode is unique to each microdroplet, i.e. same within one microdroplet but different between microdroplets. In certain embodiments, the primer member on the primer vehicle further comprises a barcode and a universal or random sequence. The primer species may be designed for targeting a specific sequence. The primer species may be a random sequence. In some cases it will be advantageous for the primers to further comprise molecular identifiers, barcodes, or to have common sequence. In certain embodiments, the primer member on the primer vehicle is tri-partite, with a universal tail portion (e.g., oligonucleotide sequences for use in sequencing library construction) immediately 5′ to a barcode sequence, followed by one of a set of random hexamer bases that enable priming from multiple places in the genome.

In certain embodiments, the method further comprises the following steps before the forming step:
providing a first solution comprising a nucleic acid template molecule;
providing a second solution comprising a plurality of different primer species each specific for a different target site on a nucleic acid template;
merging the first and second solution to form a merged solution; and
partitioning the merged solution in an immiscible carrier.

In certain embodiments, the method further comprises introducing a barcode to the microdroplets. In certain embodiments, the introducing comprises merging one of the microdroplets with a microdroplet comprising a barcode before the sequencing step.

In certain embodiments, the sequencing step is sequencing-by-synthesis. In certain embodiments, the amplifying step is carried out by polymerase chain reaction. In certain embodiments, the amplifying step is carried out by extending one or more primer species.

In certain embodiments, the nucleic acid template molecule is associated with cancer. In certain embodiments, the nucleic acid template molecule is associated with breast cancer. In certain embodiments, the nucleic acid template molecule is associated with BRCA-1 and/or BRCA-2.

In certain embodiments, the microparticle is a solid bead. In certain embodiments, the microparticle is a magnetic bead. In certain embodiments, the microparticle is a Streptavidin magnetic bead. In certain embodiments, the microparticle is a gel bead.

The provided libraries and methods have several advantages: (1) by randomly inclusion of microparticles into droplets, the highly uniform distribution of primer species over all the droplets most likely leads to existence of droplets having positive amplification reaction for any target; (2) the process is convenient and efficient without droplet merging; (3) The efforts in bioinformatics primer design can be eliminated or minimized.

Further provided herein is a kit comprising a plurality of microdroplets as described herein. In another aspect, also provided herein is a kit comprising one or more primer vehicles as described herein. The kit can also include packaging information describing the use of the microdroplets and/or microparticles.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 120 appears first in FIG. 1). All of these conventions, however, are intended to be typical or illustrative, rather than limiting.

FIG. 1 is a functional block diagram of one embodiment of a system for droplet generation and detection.

FIG. 2 is a simplified graphical representation of one embodiment of a microfluidic droplet generation device of the system of FIG. 1.

FIGS. 3A-C show a simplified graphical representation of one embodiment of a strategy for producing primer delivery vehicles and delivering into compartments.

FIG. 4 is a simplified graphical representation of one embodiment of a strategy for producing hydrogel particles for transport of primer species into compartments.

FIG. 5 is a simplified graphical representation of one embodiment of a chemical reaction for producing polymer hydrogel particles.

FIGS. 6A-C show a model digital PCR reaction for observation of SMNc.88 amplicon carried out to evaluate PCR reaction compatibility with the bead technology. Two clusters WT and NT are identified in both the control sample and when beads are loaded at 28 beads/droplet and 56 beads/droplet. This confirms the beads are compatible with the PCR amplification reaction.

FIG. 7 shows exemplified preparation of Primer Vehicles from 1 and 3 micron super-paramagnetic beads with a bound primer. Starting total primer (˜50 bp) concentration, 6.4 uM, Concentration measured by UV/Vis spectrophotometry (NanoDrop) as 110 ng/ul (which equals ˜6.5 uM). Appearance of the wild type (WT) cluster indicates presence of PCR products.

FIGS. 8A-C show an exemplified model digital PCR reaction for observation of SMNc.88 amplicon carried out to evaluate PCR reaction compatibility with superparamagnetic primer vehicle bead technology. FIG. 8A shows the control PCR solution with no beads. FIG. 8B shows the PCR solution with about 28 beads per microdroplet. FIG. 8C shows the PCR solution with about 56 beads per microdroplet. For FIGS. 8A-8C, 100uL is divided equally to four 25 uL solutions for four tests.

FIG. 9 shows images of microchannels having droplets comprising the bead solutions of FIG. 8.

FIGS. 10A and 10B show another exemplified model digital PCR reaction for observation of SMNc.88 amplicon carried out to evaluate PCR reaction compatibility with the bead technology.

FIG. 11 shows 2020 heavily overlapped targets in human genome amplified in emulsion and sequenced sequenced (Illumina MiSeq). A subset of the primer pairs (30 plex, 60 plex, and 125 plex) for a subset of the 2020 targets were either directly added into PCR solution (control) or were delivered by beads as 5-plexs on each bead type. The PCR solution were then prepared into 5 pL droplet emulsion for PCR reactions. For the sample with bead-primer delivery, since every droplet contains a limited number of beads (6, 12 or 25 beads per droplet), a droplet will have a random set of 30, 60 or 125 primer pairs (corresponding to 6, 12 or 25 beads per droplet with each bead delivering 5 primer pairs) . The random distribution mitigates the primer-primer interaction and target overlap problem. While the control experiment with primers that are not bound to any beads, gave no mapping for all the 2020 targets on Illumina sequencer, the sample with bead primer delivery gave satisfactory mapping number for more than 90 percent targets. The table shows percentage of targets that were covered with mapping number of more than 1, 15, 30, 100 and 200.

FIG. 12 shows an exemplified design of primer vehicles as provided herein.

FIG. 13 shows an exemplified synthesis of the primer vehicles from two microparticles: polymer A and polymer B; with two primers: Primer —F and Primer-B. The polymers can be natural or synthesized. In certain embodiments, the polymers can be a natural or synthesized oligo.

FIG. 14 shows an exemplified generation of multiplex primer vehicles related to BRCA-1 and BRCA-2.

FIG. 15 shows the binding capacity of the exemplified primer vehicles.

FIG. 16 shows the stability of the exemplified primer vehicle library. Primer exchange during bead storage as depicted in the FIG. 16 is expected to have a deleterious effect on performance of the Primer Vehicles. Measuring the concentration of primer release into solution when beads are stored for 3 weeks at 4 deg C is found to be a low (0.3 ng/uL). This indicates that collections of beads can be stored at 4 deg C for long periods of time. After storage, a high concentration of 20 ng/uL is released from the beads when they are heated to 90 deg C.

FIGS. 17A-D show images of an exemplified primer vehicle library.

FIG. 18 shows an exemplified design of the primer vehicle library by varying primer pair type, primer vehicle type, and microdroplet type.

FIG. 19 shows the sequencing results for a panel of 122 primer pairs that tile across contiguous regions of the genome on the BRCA1 and BRCA2 genes; all exons are covered. The results table is divided into two portions for the cases of “bead delivery of primers” and “no beads.” The “bead delivery of primers” case utilizes an exemplar primer vehicle as taught using the methods of this patent. In this case, a given primer vehicle carries a single primer pair and there are 122 different types of primer vehicles combined with the sample and master mix. Droplets (5 pL in volume) were generated at a bead concentration such that roughly 25 beads were loaded on average in each droplet. The high multiplex increases the likelihood that an amplifiable molecule is present in a given reaction. For ease of comparison between samples, the mean depth of coverage was down sampled to 2500 for all samples. The high coverage at 500×, greater than 99%, indicates exceptional uniformity of the sequencing coverage for the bead delivery with random multiplexing. In the case of the samples where all primers were present and beads and droplets were not used, the uniformity of the coverage was impacted and only 50 to 60% of the target regions were covered at a depth of 500×.

### DETAILED DESCRIPTION

As will be described in greater detail below, embodiments of the described invention include systems, methods, and kits for controlled distribution of reagents into droplets using efficient and inexpensive approaches.

### a. General

Unless otherwise defined, all technical and scientific terms used herein have the same meaning as commonly understood by one of ordinary skill in the art to which this invention belongs. Methods and materials similar or equivalent to those described herein can be used in the practice of the present invention, and exemplified suitable methods and materials are described below. For example, methods may be described which comprise more than two steps. In such methods, not all steps may be required to achieve a defined goal and the invention envisions the use of isolated steps to achieve these discrete goals. In addition, the materials, methods, and examples are illustrative only and not intended to be limiting.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C=C double bond for which the stereochemistry is not specified (e.g., —CH=CHCH₃ or

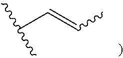

may be an (E)- or (Z)— double bond.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 7C electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(=O)R^{aa}, C(=O)N(R^{cc})₂, —CO₂R^{aa}, —SO₂R^{aa}, —C(=NR^{cc})R^{aa}, —C(=NR^{cc})OR^{aa}, —C(=NR^{cc})N(R^{cc})₂, —SO₂N(R^{cc})₂, —SO₂R^{cc}, —SO₂OR^{cc}, —SOR^{aa}, —C(=S)N(R^{cc})₂, —C(=O)SR^{cc}, C(=S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa} , R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(=O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(=O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl- [9-(10,10-dioxo- 10, 10, 10, 10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3, 5-di-t-butylphenyl)- 1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-l-cyclopropylmethyl carbamate, 1-methyl-1-(3,5-dimethoxyphenyl)ethyl carbamate, 1-methyl-1-(p-phenylazophenyl)ethyl carbamate, 1-methyl-l-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., S(=O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

Droplets can be generated using microfluidic systems or devices. As used herein, the “micro-” prefix (for example, as “microchannel” or “microfluidic”), generally refers to elements or articles having widths or diameters of less than about 1 mm, and less than about 100 microns (micrometers) in some cases. In some cases, the element or article includes a channel through which a fluid can flow. Additionally, “microfluidic”, as used herein, refers to a device, apparatus or system that includes at least one microscale channel.

A “microdroplet” according to the invention generally includes an amount of a first sample fluid encased in a second carrier fluid or a solid container or surface. Any technique known in the art for forming droplets may be used with methods of the invention. An exemplary method involves flowing a stream of the sample fluid containing the target material (e.g., nucleic acid template) such that it intersects two opposing streams of flowing carrier fluid. The carrier fluid is immiscible with the sample fluid. Intersection of the sample fluid with the two opposing streams of flowing carrier fluid results in partitioning of the sample fluid into individual sample droplets containing the target material. In some cases, the droplets may be spherical or substantially spherical; however, in other cases, the droplets may be non-spherical, for example, the droplets may have the appearance of “blobs” or other irregular shapes, for instance, depending on the external environment. In some embodiments, a droplet is a first fluid completely surrounded by a second fluid. As used herein, a first entity is “surrounded” by a second entity if a closed loop can be drawn or idealized around the first entity through only the second entity (with the sometimes exception for portions of the first fluid that may be in contact with a wall or other boundary, where applicable).

The terms “biological molecule” refers to any molecule that is present in living organisms, including large macromolecules such as proteins, carbohydrates, lipids, and nucleic acids, as well as small molecules such as primary metabolites, secondary metabolites, and natural products. In certain embodiments, the biological molecule is a protein. In certain embodiments, the biological molecule is a nucleic acid. In certain embodiments, the biological molecule is a DNA. In certain embodiments, the biological molecule is an RNA.

The term “binding moiety,” as used herein, refers to a chemical group or molecule covalently linked to a molecule, for example, a nucleic acid, and a chemical group or moiety, for example, a click chemistry handle. In some embodiments, the binding moiety is positioned between, or flanked by, two groups, molecules, or moieties and connected to each one via a covalent bond, thus connecting the two. In some embodiments, the binding moiety is an amino acid or a plurality of amino acids. In some embodiments, the binding moiety comprises 1, 2, 3, 4, 5, 6, 7, 8, 9, 10, 11, 12, 13, 14, 15, 16, 17, 18, 19, 20, or more than 20 amino acids. In some embodiments, the binding moiety comprises a poly-alanine sequence. In some embodiments, the binding moiety comprises a non-protein structure. In some embodiments, the binding moiety is an organic molecule, group, polymer, or chemical moiety. In some embodiments, the binding moiety comprises an oligonucleotide. In certain embodiments, the oligonucleotide is complementary to a primer species. In some embodiments, the binding moiety comprises a poly(T) sequence.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule. “Natural” nucleotide species include, e.g., adenine, guanine, cytosine, uracil, and thymine. Modified versions of the above natural nucleotide species include, without limitation, alpha-thio-triphosphate derivatives (such as dATP alpha S), hypoxanthine, xanthine, 7-methylguanine, 5, 6-dihydrouracil, and 5-methylcytosine.

The term “primer,” “primer species,” and “primer member” are used herein interchangeably and refer to an oligonucleotide that acts as a point of initiation of DNA or RNA synthesis under conditions in which synthesis of a primer extension product complementary to a nucleic acid strand is induced in an appropriate buffer at a suitable temperature. In certain embodiments, a primer species is an oligonucleotide. In certain embodiments, a primer species is a single stranded oligodeoxyribonucleotide. In certain embodiments, the primer species comprises a random sequence. In certain embodiments, the primer species comprises a barcode. In certain embodiments, the primer species comprises a universal sequence. In certain embodiments, the primer species comprises a barcode and a random sequence (e.g. a random hexamer). In certain embodiments, the primer species comprises a barcode and a universal sequence. The universal sequence can be used for subsequent sequencing. In certain embodiments, the primer can incorporate one or more synthetic or modified bases.

The term “variant” or “allele” as used herein generally refers to one of a plurality of species each encoding a similar sequence composition, but with a degree of distinction from each other. The distinction may include any type of variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The terms “nucleic acid template”, “nucleic acid template molecule”, “target nucleic acid template molecule”, “template nucleic acid”, “template molecule”, “target nucleic acid”, or “target molecule,” as used herein interchangeably and generally refer to a nucleic acid sequence comprising a sequence of interest that is the subject of amplification and detection processes. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid template molecule” refers to individual nucleic acid residues (e.g. nucleotides and/or nucleosides). In some embodiments, “nucleic acid template molecule” refers to an oligonucleotide chain comprising three or more individual nucleotide residues.

In some embodiments, “nucleic acid template molecule” encompasses RNA as well as single and/or double-stranded DNA. The nucleic acid template molecule may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid template molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acid template molecules can be purified from natural sources, produced using recombinant expression systems, chemically synthesized, and, optionally, purified. Where appropriate, e.g., in the case of chemically synthesized molecules, nucleic acids can comprise nucleoside analogs such as analogs having chemically modified bases or sugars, and backbone modifications. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid molecules can be obtained from an animal, plant, bacterium, fungus, viral particles or preparations, or any other biological organism. In certain embodiments, the nucleic acid molecules isolated from a single cell, tissue comprising many cells, or from cell free samples. Nucleic acid molecules can be obtained from an organism or from a biological sample obtained from an organism, e.g., from blood, urine, cerebrospinal fluid, seminal fluid, saliva, sputum, stool and tissue. Nucleic acid molecules can also be isolated from cultured cells, such as a primary cell culture or a cell line. The cells or tissues from which template nucleic acids are obtained can be infected with a virus or other intracellular pathogen.

Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures).

As used herein, the terms “oligonucleotide”, “oligo,” and “polynucleotide” can be used interchangeably to refer to a polymer of nucleotides (e.g., a string of at least three nucleotides).

The terms “digital polymerase chain reaction”, “digital PCR”, or “dPCR” as used herein generally refer to a precise method to clonally amplify and quantify nucleic acids including DNA, cDNA, or RNA by partitioning target nucleic acids into a large number of separate compartments inside of which the target nucleic acid is amplified and detected.

The term “read” or “sequence read” as used herein generally refers to data comprising the entire sequence composition obtained from a single nucleic acid template molecule or a population of a plurality of substantially identical copies of the template nucleic acid molecule.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

Some exemplary embodiments of systems and methods associated with sample preparation and processing, generation of data, and analysis of data are generally described below, some or all of which are amenable for use with embodiments of the presently described invention. In particular, the exemplary embodiments of systems and methods for preparation of nucleic acid template molecules, amplification of template molecules, detection of template molecules and/or substantially identical copies thereof. Embodiments that execute methods of detection such as digital PCR and/or sequencing methods utilizing exemplary instrumentation and computer systems are described.

Typical embodiments of “emulsions” include creating a stable emulsion of two immiscible substances, and in the embodiments described herein generally refer to an emulsion of aqueous droplets in a continuous oil phase within which reactions may occur. In particular, the aqueous droplets of an emulsion amenable for use in methods for conducting reactions with biological samples and detecting products may include a first fluid, such as a water based fluid (typically referred to as “aqueous” fluid) suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, fluorinated oils, partially fluorinated oils, or perfluorinated oils.

The term “microparticle” refers to small discrete particles. In certain embodiments, the microparticle is a bead. In certain embodiments, the microparticle is a hydrogel. The composition of the beads will vary, depending on the class of oligonucleotide and the method of synthesis. Suitable beads include those used in peptide, nucleic acid and organic moiety synthesis, including, but not limited to, plastics, ceramics, glass, polystyrene, methylstyrene, acrylic polymers, paramagnetic materials, thoria sal, carbon graphite, titanium dioxide, latex or cross-linked dextrans such as Sepharose, cellulose, nylon, cross-linked micelles and Teflon. “Microsphere Detection Guide” from Bangs Laboratories, Fishers Ind. is a helpful guide. It is to be understood that the microparticle need not be spherical; irregular microparticles may be used. In addition, the beads may be porous, thus increasing the surface area of the bead available for either capture probe attachment or tag attachment. The bead sizes range from nanometers, i.e. 100 nm, to millimeters, i.e. 1 mm, with beads from about 0.2 micron to about 200 microns being preferred, and from about 0.5 to about 5 micron being particularly preferred, although in some embodiments smaller beads may be used.

The primer species can be bound to the microparticle by approaches including, but not limited to, chemical or affinity capture (for example, including the incorporation of derivatized nucleotides such as AminoLink or biotinylated nucleotides that can then be used to attach the primer species to a surface, as well as affinity capture by hybridization), cross-linking, and electrostatic attachment, etc. In a preferred embodiment, affinity capture is used to bind the primer species to the microparticle through a binding moiety. In addition, the primer species may be biotinylated (for example using enzymatic incorporate of biotinylated nucleotides, for by photoactivated cross-linking of biotin). Biotinylated primer species can then be captured on streptavidin-coated substrate or beads, as is known in the art. Alternatively, chemical groups can be introduced to the primer species, that can them be used to add the primer species to the microparticle. In certain embodiments, microparticle has a binding moiety comprising oligo-dT.

The term “Click Reaction” means a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “Click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide—alkyne Huisgon cycloaddition; thiol-yne addition; imine formation; and Michael additions (e.g., maleimide addition).

One example of an aqueous fluid compatible with embodiments of the invention may include an aqueous buffer solution, such as ultrapure water (e.g., 18 mega-ohm resistivity, obtained, for instance by column chromatography), 10 mM Tris HC1 and 1 mM EDTA (TE) buffer, phosphate buffer saline (PBS) or acetate buffer. In the presently described example, any liquid or buffer that is physiologically compatible with nucleic acid molecules or encapsulated biological entity can be used. Also, in the same or alternative example a carrier fluid compatible with embodiments of the invention includes a non-polar solvent, decane (e g., tetradecane or hexadecane), fluorocarbon oil, silicone oil or another oil (for example, mineral oil). In certain embodiments, the carrier fluid may contain one or more additives, such as agents which increase, reduce, or otherwise create non-Newtonian surface tensions (surfactants) and/or stabilize droplets against spontaneous coalescence on contact.

Embodiments of surfactants that act to stabilize emulsions, which may be particularly useful for embodiments that include conducting reactions with biological samples such as PCR may include one or more of a silicone or fluorinated surfactant. For example, in microfluidic embodiments the addition of one or more surfactants can aid in controlling or optimizing droplet size, flow and uniformity, for example by reducing the shear force needed to extrude or inject droplets into an intersecting channel This can affect droplet volume and periodicity, or the rate or frequency at which droplets break off into an intersecting channel Furthermore, the surfactant can serve to stabilize aqueous emulsions in fluorinated oils and substantially reduce the likelihood of droplet coalescence.

In some embodiments, the aqueous droplets may be coated with a surfactant or a mixture of surfactants, where those of skill in the art understand that surfactant molecules typically reside at the interface between immiscible fluids, and in some cases form micelles in the continuous phase when the concentration of surfactant(s) is greater than what is referred to as the critical micelle concentration (also sometimes referred to as CMC). Examples of surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglycerl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

In one embodiment, a fluorosurfactant can be prepared by reacting the perflourinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Flourinert (3M)), which then serves as the carrier fluid (e.g. continuous phase). In the presently described embodiment, the surfactant produced is an ionic salt, and it will be appreciated that other embodiments of non-ionic surfactant compositions may also be used. For example, non-ionic surfactant composition may include what are referred to as block copolymers (e.g. di-block, or tri-block copolymers) typically comprising a head group and one or more tail groups. A more specific example of a fluorinated block copolymer includes a polyethylene glycol (PEG) head group and one or more perfluoropolyether (PFPE) tail groups.

Further, in some embodiments other reagents that act as droplet stabilizers (also referred to as passivating agents) may be included. Useful droplet stabilizing reagents may include, but are not limited to, polymers, proteins, BSA, spermine, or PEG.

In some embodiments, desirable characteristics may be achieved by adding a second surfactant, or other agent, such as a polymer or other additive, to the aqueous fluid. Further, in certain embodiments utilizing microfluidic technology the carrier fluid may be caused to flow through the outlet channel so that the surfactant in the carrier fluid coats the channel walls.

In the embodiments described herein, droplets of an emulsion may be referred to as partations, compartments, microcapsules, microreactors, microenvironments, or other name commonly used in the related art. The aqueous droplets may range in size depending on the composition of the emulsion components or composition, contents contained therein, and formation technique employed. The described emulsions are microenvironments within which chemical reactions that may include binding reactions, Reverse Transcription, PCR, or other process may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a microparticle such as a bead or hydrogel. In some embodiments, beads may be employed for attachment of template and amplified copies of the template, amplified copies complementary to the template, or combination thereof. Further, the substrate may be enabled for attachment of other type of nucleic acids, reagents, labels, or other molecules of interest. It will also be appreciated that the embodiments described herein are not limited to encapsulating nucleic acids in droplets, but rather the droplets may be configured to encapsulate a variety of entities that include, but are limited to, cells, antibodies, enzymes, proteins, or combinations thereof. As with nucleic acids, the droplets may further be amenable to performing various reactions on the entities encapsulated therein and/or detection methods such as, for instance, ELISA assays.

Various methods of forming emulsions may be employed with the described embodiments. In the some embodiments methods involve forming aqueous droplets where some droplets contain zero target nucleic acid molecules, some droplets contain one target nucleic acid molecule, and some droplets may contain multiple target nucleic acid molecules. It will be appreciated by those of skill in the art that in some embodiments it may be desirable for individual droplets to contain multiple nucleic acid molecules from a sample, however in certain assays there may be a discrete number of targets of interest where droplets are generated based on the likelihood that there is at most a single target of interest in each droplet in the presence of other nucleic acid molecules that are not targets of interest.

In some embodiments the number of target nucleic acid molecules in the droplets is controlled via a limiting dilution of the target nucleic acid molecules in the aqueous solution. Alternatively, in some embodiments the number of target nucleic acid molecules in the droplets is controlled via a method of partitioning very small volumes of the aqueous fluid (e.g. picoliter—nanoliter volumes such as a volume of about 5 picoliters) into the droplet where the statistical likelihood of distributing multiple target nucleic acid molecules in the same droplet is very small. In some or all of the described embodiments, the distribution of molecules within droplets can be described by Poisson distribution. However, it will be appreciated that methods for non-Poisson loading of droplets may be employed in some embodiments and include, but are not limited to, active sorting of droplets such as by laser-induced fluorescence, or by passive one-to-one loading.

Systems and methods for generation of emulsions include what are referred to as “bulk” emulsion generation methods that generally include an application of energy to a mixture of aqueous and carrier fluids. In the example of bulk generation methods energy may be applied by agitation via vortexing, shaking, spinning a paddle (to create shear forces) in the combined mixture or in some embodiments the agitation of the aqueous solution may applied when separate from the immiscible fluid where the agitation results in droplets being added to the immiscible fluid as for example when piezo-electric agitation is employed. Alternatively, some bulk generation methods include adding the aqueous fluid drop-wise to a spinning carrier fluid. Bulk emulsion generation methods typically produce emulsions very quickly and do not require complicated or specialized instrumentation. The droplets of the emulsions generated using bulk generation techniques typically have low uniformity with respect to dimension and volume of the droplets in the emulsion.

Other embodiments of emulsion formation methods include “microfluidic” based formation methods that may employ a junction of channels carrying aqueous and carrier fluids that result in an output of droplets in a stream of flow. Some embodiments of microfluidic based droplet generation approaches may utilize one or more electric fields to overcome surface tension. Alternatively, some embodiments do not require the addition of an electric field. For example, a water stream can be infused from one channel through a narrow constriction; counter propagating oil streams (preferably fluorinated oil) hydrodynamically focus the water stream and stabilize its breakup into droplets as it passes through the constriction. In order to form droplets, the viscous forces applied by the oil to the water stream must overcome the water surface tension. The generation rate, spacing and size of the water droplets is controlled by the relative flow rates of the oil and the water streams and nozzle geometry. While this emulsification technology is extremely robust, droplet size and rate are tightly coupled to the fluid flow rates and channel dimensions.

Continuing with the present example, some embodiments of microfluidic devices of can incorporate integrated electric fields, thereby creating an electrically addressable emulsification system. For instance, this can be achieved by applying high voltage to the aqueous stream and charge the oil water interface. The water stream behaves as a conductor while the oil is an insulator; electrochemical reactions charge the fluid interface like a capacitor. At snap-off, charge on the interface remains on the droplet. The droplet size decreases with increasing field strength. At low applied voltages the electric field has a negligible effect, and droplet formation is driven exclusively by the competition between surface tension and viscous flow

Additional examples of systems and methods for forming aqueous droplets surrounded by an immiscible carrier fluid in microfluidic structures are described U.S. Pat. Nos. 7,708,949; and 7,041,481 (reissued as RE 41,780) and U.S. Published Patent application Ser. Nos. 2006/0163385 A1; 2008/0014589; 2008/0003142; and 2010/0137163; and 2010/0172803 each of which is hereby incorporated by reference herein in its entirety for all purposes.

In some embodiments, emulsion formation methods also include merging already formed emulsion droplets with other droplets or streams of fluid to produce combined droplets. The merging of droplets can be accomplished using, for example, one or more droplet merging techniques described for example in Link et al. (U.S. patent application numbers 2008/0014589; 2008/0003142; and 2010/0137163) and European publication number EP2047910 to Raindance Technologies Inc.

In certain embodiments, a reverse transcriptase reaction (referred to as an “RT” reaction) may be used to convert from RNA starting material to a nucleic acid such as cDNA or other synthetic nucleic acid derivative. Reverse transcriptase reaction refers to methods known in the art, for example by methods described by Yih-Horng Shiao, (BMC Biotechnology 2003, 3:22; doi:10.1186/1472-6750-3-22). See also J Biomol Tech. 2003 March; 14(1): 33-43, which includes a discussion of RT reaction methods, each of which is incorporated by reference. For example, the process includes a first step of introducing a reverse transcriptase enzyme used to generate single stranded complementary DNA (cDNA) from an RNA template using target-specific primers (sometimes referred to as “RT primers”), random hexamers, or poly-alanine tail targeting oligonucleotide. For embodiments of conversion of small RNA to cDNA a target-specific stem loop primer may be used to add length and optimize characteristics such as melting temperature and specificity. In some embodiments, the single stranded cDNA is then used as a template for conversion of a second strand complementary to the single stranded cDNA. The single or double stranded cDNA may then be used as a template for amplification, such as by PCR. The process for amplifying the target sequence can include introducing an excess of oligonucleotide primers to a DNA or cDNA mixture containing a desired target sequence, followed by a precise sequence of thermal cycling in the presence of a DNA polymerase. The primers are complementary to their respective strands of the double stranded target sequence.

As described elsewhere in this description, the described embodiments include conducting reactions with biological entities within the emulsion droplets. An example of a very useful class of reactions includes nucleic acid amplification methods. The term “amplification” as used herein generally refers to the production of substantially identical copies of a nucleic acid sequence (typically referred to as “amplicons”). One of the most well-known amplification strategies is the polymerase chain reaction (e.g., Dieffenbach and Dveksler, PCR Primer, a Laboratory Manual, Cold Spring Harbor Press, Plainview, N. Y. [1995]). The amplification reaction may include any amplification reaction known in the art that amplifies nucleic acid molecules, such as Loop-mediated Isothermal Amplification (also referred to as LAMP), Helicase-dependent amplification (HDA), Nicking enzyme amplification reaction (NEAR), polymerase chain reaction, nested polymerase chain reaction, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification (SDA), transcription based amplification system, nucleic acid sequence-based amplification, rolling circle amplification, and hyper-branched rolling circle amplification.

In some embodiments, generally referred to as “multiplexing”, emulsion droplets comprise a plurality of species of primer pairs each specific to amplify a different region of nucleic acid sequence. Optimization of traditional multiplexing of standard PCR primers in tubes or wells is known to be difficult. Multiple PCR amplicons being generated in the same reaction can lead to competition between amplicons that have differing efficiencies due to differences in sequence or length or access to limiting reagents. This results in varying yields between competing amplicons which can result in non-uniform amplicon yields. However, because droplet based digital amplification utilizes only one template molecule per droplet, even if there are multiple PCR primer pairs present in the droplet, only one primer pair will be active. Since only one amplicon is being generated per droplet, there is no competition between amplicons or reagents, resulting in a more uniform amplicon yield between different amplicons.

In some embodiment, even though the number of PCR primer pairs per droplet is greater than one, there is still at most only one template molecule per droplet and thus there is only one primer pair per droplet that is being utilized at one time. This means that the advantages of droplet amplification for eliminating bias from either allele specific PCR or competition between different amplicons is maintained.

Additional examples describing systems and methods for performing amplification in droplets are shown for example in Link et al. (U.S. patent application numbers 2008/0014589, 2008/0003142, and 2010/0137163), Anderson et al. (U.S. patent number 7,041,481 and which reissued as RE 41,780) and European publication number EP2047910 to Raindance Technologies Inc. The content of each of which is incorporated by reference herein in its entirety.

In certain cases it is desirable to release the contents of the droplets to use in further processing and/or detection processes. In some embodiments, the contents of many droplets are released and pooled together, however it will be appreciated that in some embodiments the contents of droplets are released individually and maintained separately. Various methods for releasing the contents of droplets may be employed, typically depending on the composition of the droplets. For example, in cases where aqueous droplets are in a silicone based oil an organic solvent may be used to “break” the integrity of the interface between the aqueous fluid and silicone oil combining into a single solution that may be separated using various techniques. Alternatively, in cases where aqueous droplets are in a fluorinated oil a perfluorinated alcohol reagent may be used. In the present example, the perfluorinated alcohol provides advantages for use as a releasing agent in that it is not immiscible with aqueous fluid (e.g. will not be present in aqueous phase post release) and works very well to disrupt surfactants typically used with fluorinated oils. One specific example of perfluorinated alcohol useful for release applications includes perfluoro decanol.

In some embodiments, often referred to as digital PCR, after amplification the emulsion droplets are introduced into an instrument for optical detection of amplification products. In some embodiments the generation and amplification of the nucleic acid molecules occurs in a single fluidic chip that is also used for detection, alternatively the emulsion droplets may be removed or dispensed from a fluidic chip used for droplet generation in order to conduct the amplification “off-chip”. For embodiments of the off-chip application the droplets may be introduced into either a second fluidic chip used for detection or into the original fluidic chip used for droplet generation. Further, in embodiments where the emulsion droplets are generated using bulk methods, after amplification the droplets may be introduced into a fluidic chip used for detection. In the same or alternative embodiment detection of reaction products produced from PCR thermocycling may be performed during or after each amplification cycle (e.g. sometimes referred to as “real time” PCR). The detected signals form the reaction products may be used to generate what are referred to as “melt curves” sometimes used with known concentrations as standards for calibration. Melt curves may also be based on the melting temperature of probes in the reaction where combinations of probes are associated with specific sequence composition of a target (e.g. as an identifier or type of molecular barcode) where the presence of the target can be identified from the melt curve signature.

In some embodiments, when droplets are introduced into a fluidic chip used for detection it may be highly desirable to add additional carrier fluid to increase the spacing between successive droplets. Examples of increasing the spacing between droplets is described in US Patent Application Ser. No. 2010-0137163, which is hereby incorporated by reference herein in its entirety for all purposes.

The emulsion droplets may be individually analyzed and detected using any methods known in the art, such as detecting the presence and/or amount of signal from a reporter. Generally, the instrument for detection comprises one or more detection elements. The detection elements can be optical, magnetic, electromagnetic, or electrical detectors, other detectors known in the art, or combinations thereof. Examples of suitable detection elements include optical waveguides, microscopes, diodes, light stimulating devices, (e.g., lasers), photo multiplier tubes, charge-coupled devices (CCD), and processors (e.g., computers and software), and combinations thereof, which cooperate to detect a signal representative of a characteristic, marker, or reporter. Further description of detection instruments and methods of detecting amplification products in droplets are shown in Link et al. (U.S. patent application Nos. 2008/0014589, 2008/0003142, and 2010/0137163) and European publication number EP2047910 to RainDance Technologies Inc., each of which is hereby incorporated by reference herein in its entirety for all purposes.

In certain embodiments, amplified target nucleic acid molecules are detected using detectably labeled probes, such as hybridization probes. In some or all of the described embodiments a probe type may comprise a plurality of probes that recognize a specific nucleic acid sequence composition. For example, a probe type may comprise a group of probes that recognize the same nucleic acid sequence composition where the members of the group have one or more detectable labels specific for that probe type and/or members that do not include a detectable label (that may be included to modulate intensity of reporter signal). Further the probe members may be present at different concentrations relative to each other within the droplets. Thus, the combination of detectable labels and relative intensities detected from the concentrations of probes are specific to and enable identification of the probe type. Those of ordinary skill in the related art appreciate that the embodiments described herein are compatible with any type of fluorogenic DNA hybridization probes or hydrolysis probes, such as TaqMan probes, molecular beacons, Solaris probes, scorpion probes, and any other probes that function by sequence specific recognition of target DNA by hybridization and result in increased fluorescence on amplification of the target sequence. Further in the embodiments described herein, probe types may also be multiplexed in emulsion droplets in the same way as described elsewhere with respect to multiplexing primer species.

As described elsewhere, the droplets may contain a plurality of detectable probes that hybridize to amplicons produced in the droplets. Members of the plurality of probes can each include the same detectable label, or a different detectable label. The plurality of probes can also include one or more groups of probes at varying concentration. The groups of probes at varying concentrations can include the same detectable label which varies in intensity, due to varying probe concentrations. In the embodiments described herein, the fluorescence emission from each fused droplet may be determined and plotted on a scattered plot based on its wavelength and intensity. Examples of probe detection and analysis using wavelength and intensity is described in US Patent Application Serial No 2011/0250597, which is hereby incorporated by reference herein in its entirety for all purposes.

Types of detectable labels suitable for use with probes specific to bridge regions of a primer and other probes for use in methods of the invention are described hereinafter. In some embodiments, the detectably labeled probes are optically labeled probes, such as fluorescently labeled probes. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-l-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.TM. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels for certain embodiments include FAM and VIC, and in the same or alternative embodiments may also include TET, Yakima yellow, Calcein orange, ABY and JUN dyes (from Thermo Fisher Scientific). Labels other than fluorescent labels are contemplated by the invention, including other optically-detectable labels.

Additional examples of digital amplification and detection of reporters are described in U.S. Pat. No. 8,535,889, which is hereby incorporated by reference herein in its entirety for all purposes.

In embodiments of digital PCR, data analysis typically involves a scatter plot type of representation for identifying and characterizing populations of statistically similar droplets that arise from unique probe signatures (wavelength and intensity), and for discriminating one population of droplets from the others. In some embodiments, a user and/or computer application may select data points associated with specific droplets or groups of droplets within histograms, either for counting, or for assay selection as in the use of optical labels, or for any other purpose. Some methods of selection may include the application of boundaries surrounding one or more selections, either closed or unclosed, of any possible shape and dimension.

The embodiments described herein are not limited to the use of a specific number of probe species. In some embodiments a plurality of probe species are used to give additional information about the properties of nucleic acids in a sample. For example, three probe species could be used wherein a first probe species comprises a fluorophore that has a particular excitation and emission spectra (e.g., VIC), and a second probe species comprises a fluorophore that has a particular excitation and emission spectra (e.g., FAM) where the excitation spectra for the first and second probe species may overlap but have clearly distinct emission spectra from each other. Detected differences in intensity can be used to discriminate between different probe species that employ the same fluorophore, where the intensity may be tunable of emitted light. In some of the described embodiments, a further step of releasing converted or amplified target molecules from the emulsion droplets for further analysis. The released converted or amplified material can also be subjected to further processing and/or amplification. Additional examples of systems and methods of releasing amplified target molecules from the droplets are described in Link et al. (U.S. patent application numbers 2008/0014589, 2008/0003142, and 2010/0137163) and European publication number EP2047910 to RainDance Technologies Inc.

In certain embodiments, the amplified target molecules are sequenced using any suitable sequencing technique known in the art. In one example, the sequencing is single-molecule sequencing-by-synthesis. Single-molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Other examples of sequencing nucleic acids may include Maxam-Gilbert techniques, Sanger type techniques, Sequencing by Synthesis methods (SBS), Sequencing by Hybridization (SBH), Sequencing by Ligation (SBL), Sequencing by Incorporation (SBI) techniques, massively parallel signature sequencing (MPSS), polony sequencing techniques, nanopore, waveguide and other single molecule detection techniques, reversible terminator techniques, or other sequencing technique now know or may be developed in the future.

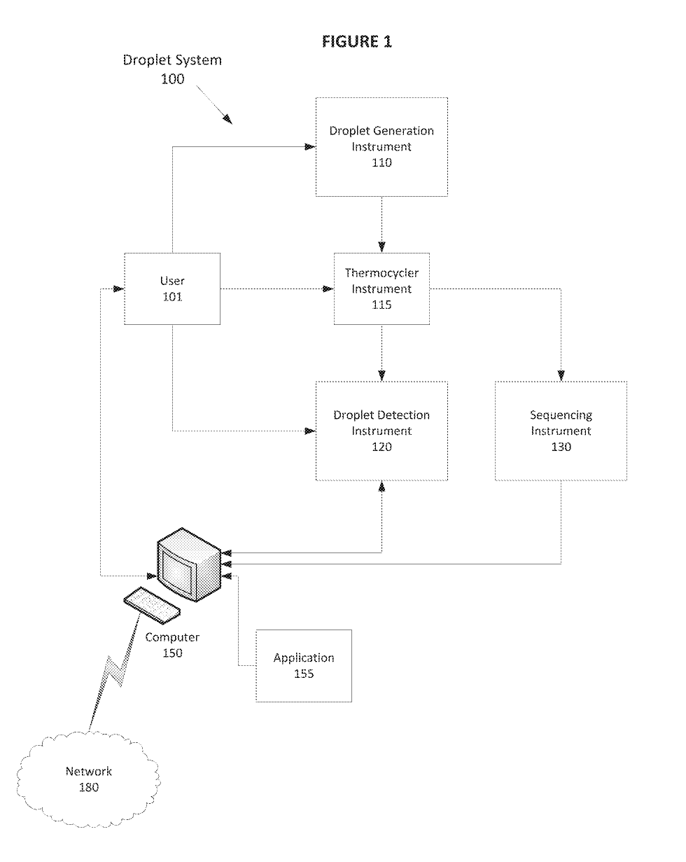

Embodiments of a typical fluidics based droplet digital amplification platform generally include one or more instrument elements employed to execute one or more process steps. FIG. 1 provides an illustrative example of droplet system 100 constructed and arranged to generate droplets containing templates, amplification of the templates, and detection of the amplified products. In some embodiments, droplet system 100 includes droplet generation instrument 110, thermocycler instrument 115, and droplet detection instrument 120, although it will be appreciated that operations may be combined into a single instrument depending on the number and nature of process steps. Importantly, user 101 may include any type of user of droplet digital amplification technologies.

Also in the same or alternative embodiments, droplet system 100 comprises sequencing instrument 130 that may include a subsystem that operatively couples a reaction substrate to a particular mode of data capture (i.e. optical, temperature, pH, electric current, electrochemical, etc.), one or more data processing elements, and a fluidic subsystem that enables execution of sequencing reactions on the reaction substrate. For example, some embodiments of detectors for fluorescence readout may include conventional epifluorescence microscopy with a custom microscope. In the present example, a 20mW, 488 nm laser source (Cyan; Picarro, Sunnyvale, CA) may be expanded 2× and focused by the objective lens (20×/0.45 NA; Nikon, Japan) onto a microfluidic channel. Two band pass filters discriminate the fluorescence collected through the objective lens: 512/25 nm and 529/28 nm for FAM and VIC fluorophores respectively (Semrock, Rochester, NY). Fluorescence may be detected by two H5784-20 photomultipliers (Hamamatsu, Japan) and is typically recorded at a 200 kHz sampling rate with a USB-6259 data acquisition card (National Instruments, Austin, Tex.).

Further, as illustrated in FIG. 1, droplet system 100 may be operatively linked to one or more external computer components, such as computer 150 that may, for instance, execute system software or firmware, such as application 155 that may provide instructional control of one or more of the instruments, such as droplet generation instrument 110, thermocycler instrument 115, droplet detection instrument 120, sequencing instrument 130, and/or signal processing/data analysis functions. Computer 150 may be additionally operatively connected to other computers or servers via network 180 that may enable remote operation of instrument systems and the export of large amounts of data to systems capable of storage and processing. Also in some embodiments network 180 may enable what is referred to as “cloud computing” for signal processing and/or data analysis functions. In the present example, droplet system 100 and/or computer 130 may include some or all of the components and characteristics of the embodiments generally described herein.

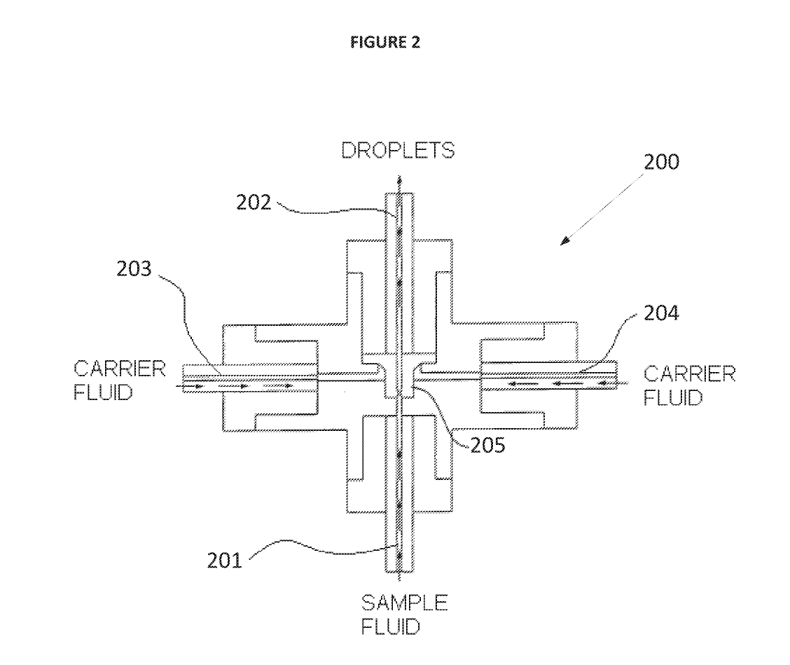

FIG. 2 provides an illustrative example of droplet generator 200. Droplet generation instrument 110 typically includes one or more embodiments of droplet generator 200, where in some embodiments it is highly desirable to have multiple embodiments of droplet generator 200 that operate in parallel to substantially increase the rate of droplet generation. In the present example, droplet generator 200 includes inlet channel 201, outlet channel 202, and two carrier fluid channels 203 and 204. Channels 201, 202, 203, and 204 meet at a junction 205. Inlet channel 201 flows sample fluid to junction 205. Carrier fluid channels 203 and 204 flow a carrier fluid that is immiscible with the sample fluid to junction 205. Inlet channel 201 narrows at its distal portion wherein it connects to junction 205. Inlet channel 201 is oriented to be perpendicular to carrier fluid channels 203 and 204. As described elsewhere, droplets are formed as sample fluid flows from inlet channel 201 to junction 205, where the sample fluid interacts with flowing carrier fluid provided to the junction 205 by carrier fluid channels 203 and 204. Outlet channel 202 receives the droplets of sample fluid surrounded by carrier fluid.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. Those of ordinary skill in the related art will appreciate that interfaces may include one or more GUI's, CLI' s or a combination thereof.

A processor may include a commercially available processor or a processor that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system that interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more experiments or processes. Additionally, an internet client may include an application enabled to accesses a remote service on another computer using a network and may for instance comprise what are generally referred to as “Web Browsers”. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that may employ what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above, embodiments of the described invention relate to systems, methods, and kits that provide an inexpensive strategy and vehicles for delivery of reagents into microfluidicly generated droplets. More specifically, various embodiments of the invention include efficient mechanisms for compartmentalizing a plurality of primer species in partitions with nucleic acids and other components necessary to conduct a reaction in the partitions. In some embodiments, the mechanisms include use of a specialized primer delivery vehicle that compartmentalize primer species content into droplets without complicated droplet merging or coalescence steps where the primer delivery vehicles do not interfere with amplification or other processing steps. For example, embodiments of the presently described invention include strategies for efficiently producing individual droplets that include a number of different primer species compartmentalized inside without the added expense of incorporating electric fields or other microfluidic structures designed to merge droplets with other droplets or fluids. In the embodiments described herein a primer delivery vehicle may be employed to transport a sufficient number of members (e.g. copies) of a primer species into a partition or compartment (e.g. a droplet, well, chamber, etc.) to enable a desired reaction. It is typically desirable that the compartments include a desired number and/or variety (e.g. multiplexed) of primer species delivered by a plurality of delivery vehicles, the individual primer members being easily separable from the delivery vehicles in sufficient concentration to support use in a reaction.

In some embodiments, it is highly desirable to have a degree of multiplexing of primer species in each droplet that statistically raises the possibility that a single nucleic acid molecule compartmentalized within the droplet includes a target region for at least one of the target species. In some embodiments, the delivery vehicles may each carry a species of primers (e.g. the species includes sense and antisense primer members, also sometimes referred to as forward and reverse primers) where multiple delivery vehicles are distributed into each droplet (e.g. a mean of 3-100 delivery vehicles per droplet, or more than 100 which may depend on factors such as droplet volume, delivery vehicle dimension, etc.). In some embodiments the distribution may be random however in alternative embodiments some degree of control of the distribution may be applied. Also, in some embodiments a moderate degree of multiplexing may be desirable to reduce the possibility of interactions between some primer species where, for instance, if the design of primer species is not certain to be free of interactions the higher the degree of multiplexing increases the possibility of two primer species being compartmentalized together that will interact with each other producing undesirable products.

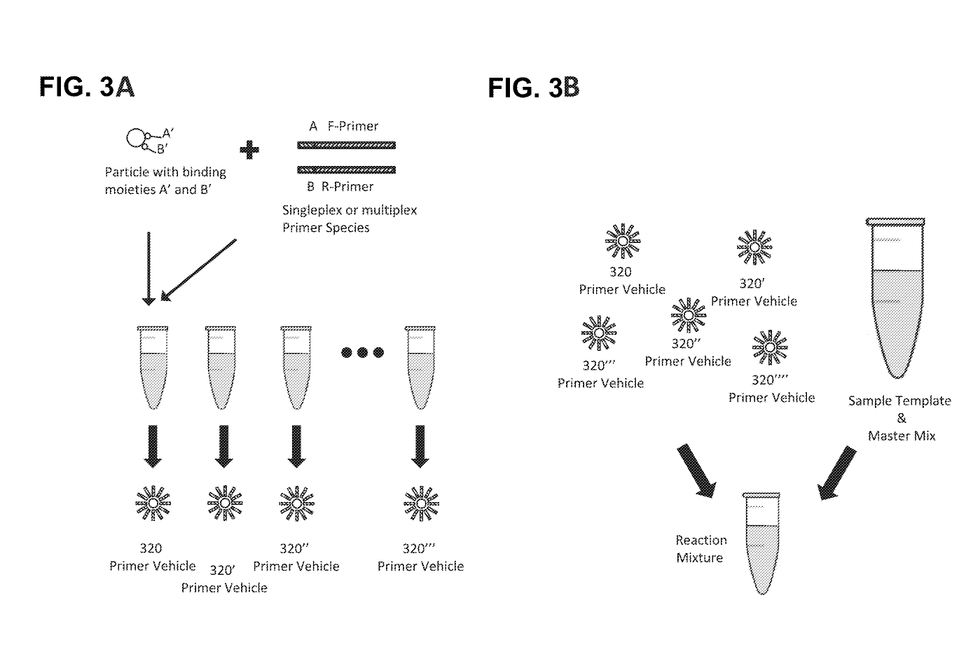

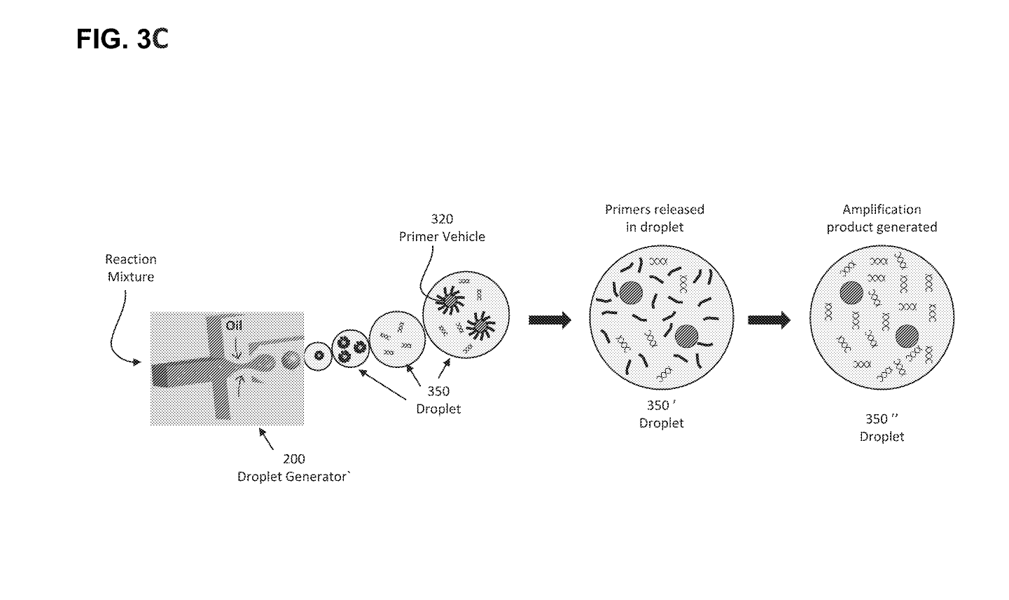

One embodiment of a primer delivery vehicle may comprise a bead type element with a linking element disposed on available surfaces (e.g. outer and/or porous surfaces). The bead element may include any type of bead known to those of ordinary skill in the related art such as a polystyrene, or agarose type bead element. It will be appreciated, however, that different types of bead elements have different characteristics that may be desirable or undesirable in certain applications. For instance, it may be desirable for the bead element to have certain heat tolerance, melting temperature, pH buffering, porosity (e.g. porous enough to allow primer access/binding within pore structures), or other characteristic that provides a useful function in the contemplated application that may include steps that occur within droplets or outside of the droplet microenvironment. Another desirable characteristic may include a small dimension of the bead relative to the dimension of the droplet, where for instance a dimension of 5 μm or less may be desirable for droplets of about 20 μm. One example of such a bead based primer delivery vehicle embodiment is illustrated in FIGS. 3A-C that includes an embodiment of bead 305 which, for example, may be composed of a hydrogel PEG material and include a coating of binding elements disposed on the surface.

Binding elements, illustrated as binding moiety 307 may include any type of binding element known in the art such as an oligonucleotide bound to the surface using standard chemistries. In embodiments where binding moiety 307 comprises an oligonucleotide, binding moiety 307 may immobilized on bead 305 and include a region that is complementary to a region of one or more primer species, typically all of the primer species to be employed illustrated as primer species 310′, 310″, and 310′″. In some embodiments each primer species is individually immobilized on an embodiment of bead 305 via hybridization of the complementary regions to produce embodiments of primer vehicle 320 (illustrated in FIGS. 3A-C as primer vehicle 320, 320′, 320″, 320′″, and 320′″ each associated with different primer species). It will also be appreciated that multiple embodiments of primer species 310 may be immobilized on a single embodiment of bead 305 to produce a multiplexed embodiment of primer vehicle 320.

It will also be appreciated that it may be desirable to attach binding moiety 307 to bead 305 by the 3′ end of binding moiety 307. In one example, binding moiety 307 may be biotinylated at the 3′ end and attached to streptavidin-functionalized embodiments of bead 305. In some embodiments, the streptavidin may provide additional binding sites for the biotin relative to those availbel on the surface of bead 305, thus increasing the number of members of primer species that can be transported by bead 305.

In the described embodiments, the complementary regions include sequence composition that has a melting temperature (Tₘ) that is higher than typical ambient temperatures, but easily releases at a desired temperature which may include a melting temperature associated with a PCR reaction. Further, in some or all of the described embodiments the complementary region is the same for all embodiments of primer species 310 so that a generic embodiment of binding moiety 307 is easily employed. However, in alternative embodiments it may be advantageous to use different embodiments of binding moiety 307 each having a different sequence composition of the complementary region which may correspond to one or more embodiments of the complementary region of primer species 310 and/or correspond to different members within primer species 310 (e.g. different sequence composition between the forward and reverse members of a primer species). The use of different embodiments of binding moiety 307 may advantageously allow for greater control of the distribution of particular embodiments of primer species 310 within a combined population and/or for the distribution of the members of primer species 310 on bead 305.

Typically, the embodiments of primer vehicle 320 are combined into receptacle 330 for storage and use in droplet generation, where receptacle 330 may include any type of receptacle known in the art that include but are not limited to tubes, cuvettes, plates, etc. The combined embodiments of primer vehicle 320 comprising the different primer species may be referred to as a “library” of primer species. In some embodiments, a library of primer vehicle 320 embodiments may be lyophilized to provide improved characteristics such as limiting the possibility of undesired dissociation of primers from primer vehicle 320, extended shelf life, etc.

In some or all of the embodiments described herein the library of primer species immobilized as primer vehicle 320 may then be mixed with nucleic acid molecules as well as all necessary reagents for performing a desired assay, such as an amplification reaction. In the described embodiments it may be desirable that primer vehicle 320 is substantially neutrally-buoyant which may typically be a function of the composition and/or modifications of bead 305. It will however also be appreciated that if necessary the mixtures may be agitated to produce or maintain a substantially homogeneous suspension (e.g. even distribution) of the embodiments of primer vehicle 320 in the mixture prior to generation of an emulsion of aqueous droplets using the mixture.

In the described embodiments, one or more embodiments of droplet generator 200 may be employed to produce a plurality of droplets from the mixture comprising the embodiments of primer vehicle 320 (illustrated in FIG. 3C as droplets 350), which typically include a number of at least 1000, 100000, 1000000, 10000000, or more droplets. The embodiments of droplet 350 typically contain a number of embodiments of primer vehicle 320 according to a Poisson distribution with a mean number of primer vehicles 320 depending on the volume of the droplet, dimension of beads 305, and the concentration of primer vehicle 320 embodiments in the mixture. For example, the droplets may have a mean number of primer vehicle 320 embodiments ranging from 3-100 in each droplet.

In the described embodiments, droplets 350 may then be exposed to a temperature greater than the melting temperature of the complementary regions between binding moiety 307 and primer species 310 resulting in a release of primer species 310 from the embodiments of primer vehicle 320 and into the aqueous environment with the droplet, illustrated in FIG. 3C as droplet 350′. Next, in some embodiment's droplet 350′ may be subjected to a thermocyling process typical of PCR reaction to produce a population of substantially identical copies of one or more regions from a nucleic acid molecule targeted by primer species 310, illustrated in FIG. 3C as droplet 350″.

Returning to the composition and characteristics of bead 305, various embodiments of bead 305 may be employed with the multiplexed delivery strategy described above. Some embodiments may include a bead functionalized to immobilize an oligonucleotide binding moiety molecule by its 3′ end so that the 5′ end is free in solution. In the same or alternative embodiment, bead 305 may be functionalized with streptavidin that provides a greater number of binding sites for biotinylated oligonucleotide binding moieties.

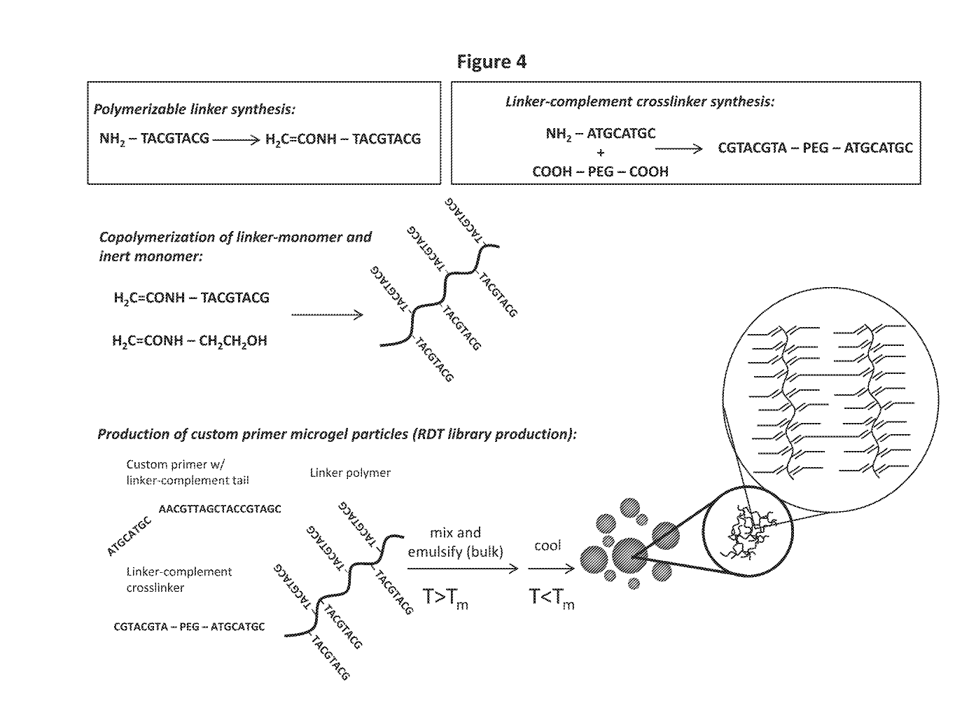

Another embodiment of bead 305 may include what is referred to as a “hydrogel particle” composed of polymer chains cross-linked by reversible bonds. In certain embodiments, the reversible bonds can be broken by a triggering event, wherein the triggering event is one or more selected from the group consisting of a chemical trigger, a biological trigger, a thermal trigger, an electrical trigger, an illuminating trigger, and/or a magnetic trigger. Further, in the embodiments described herein the polymer chains comprise moieties that reversibly couple to oligonucleotide molecules. In other words, the polymer chains link to form the hydrogel particle where the links are subsequently broken in the compartments in response to stimulus (e.g. temperature, pH, etc.) releasing the members of the primer species. FIG. 4 provides an illustrative example of material and chemical composition of one embodiment as well as an approach for producing them.

For example, there are multiple options for crosslinkable polymer chains containing reversible primer linking and crosslinking groups. Possible elements that can be combined together to achieve the material chemistry for the crosslinkable polymer chains include a soluble polymer chain that is linear, branched, dendritic, or multi-arm polymers (e.g., 4-arm PEG). In general it is desirable that the soluble polymer is water soluble and may include one or more of PEG, natural polymers (e.g. gelatin), polyacrylamide, polymers with hydrophilic pendant groups (e.g., polyHEMA).

In the described embodiments the linking moiety are optimized to achieve an effective crosslink density (dictates mechanical properties and size of the swollen microparticle gels, rate of solubilization) and maximum payload of primers. The linking moieties could be the same throughout the polymer or could be a collection of different types of moieties. For example, multiple types of binding moieties might be used for linking different types of primers and/or to control the relative concentration of different species being delivered. Complementary linking moieties could be included on the same polymer, which means that the material will be self-crosslinkable, however some of the linking groups could become involved in intramolecular interactions.

In some embodiments, a series of polymers could be functionalized with a universal linking moiety and individual binding moiety. This facilitates tuning the relative concentration of linking moieties in the polymer gel (by blending different types of polymers together) without the need to change the relative concentration of different linking sequences within a polymer chain.

In the described embodiments, it may be desirable that the sequence of the binding moiety on the primer vehicles include one or more of the following: melting temperature that is high enough to prevent particles from degrading at low temperature but low enough to facilitate dissolution and primer release and dissolution at amplification conditions; binding moiety sequence that is specific to only the primer tail sequence to prevent unwanted interference with PCR or downstream sequencing; the binding moiety can include an enzymatic or thermally-labile element so it can be “turned-off” once the primer species is delivered (e.g., dUTP). Further, the binding moiety may also include: non-covalent crosslinking chemistry, reversible interaction other than oligonucleotide hybridization. Also in some embodiments the primers might not be “linked” within the gel. For example, the crosslink density (i.e., pore size) of the gel may be tuned so that the primer payload could be physically trapped within the gel before temperature actuated degradation and release.

Various methods might be used to make the microparticles. In one possible example, the soluble polymer, members of one or more primer species, and crossbinding moiety (if needed) could be mixed together and heated above the Tm of the binding moiety chemistry. Then, the solution would be partitioned into droplets and cooled to hybridize linkages and crosslink the gel. After stabilization of the gel, then the immiscible phase is removed by filtration or other methods. In the same or alternative example, the soluble polymer could be functionalized with the primer payload. Then, a second step could be used to create individual particles from the primer-containing polymers.

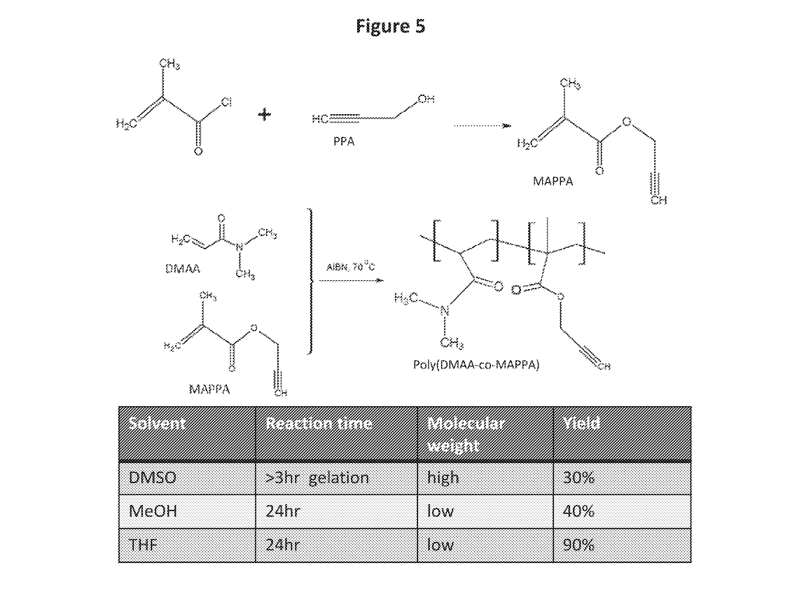

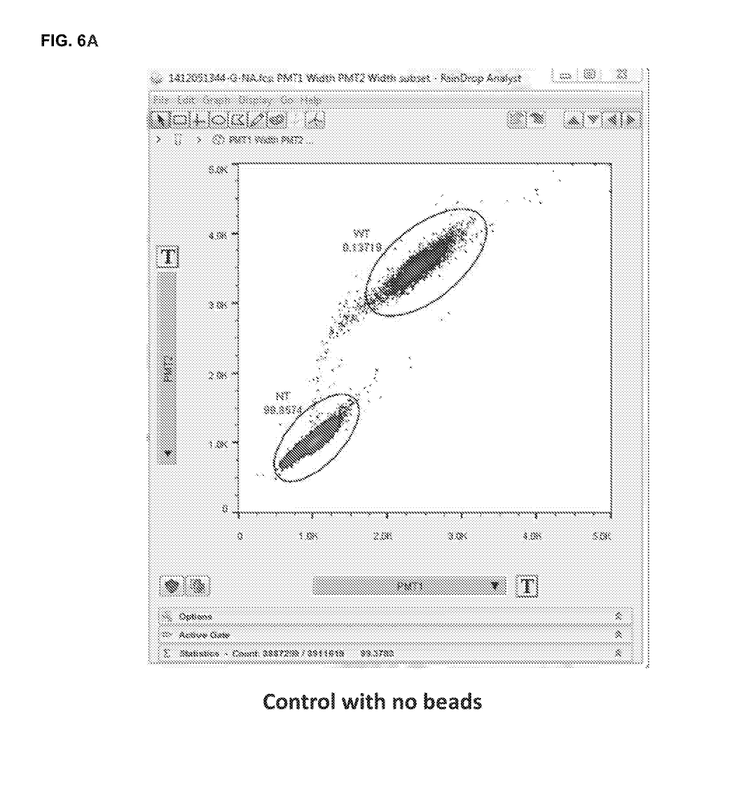

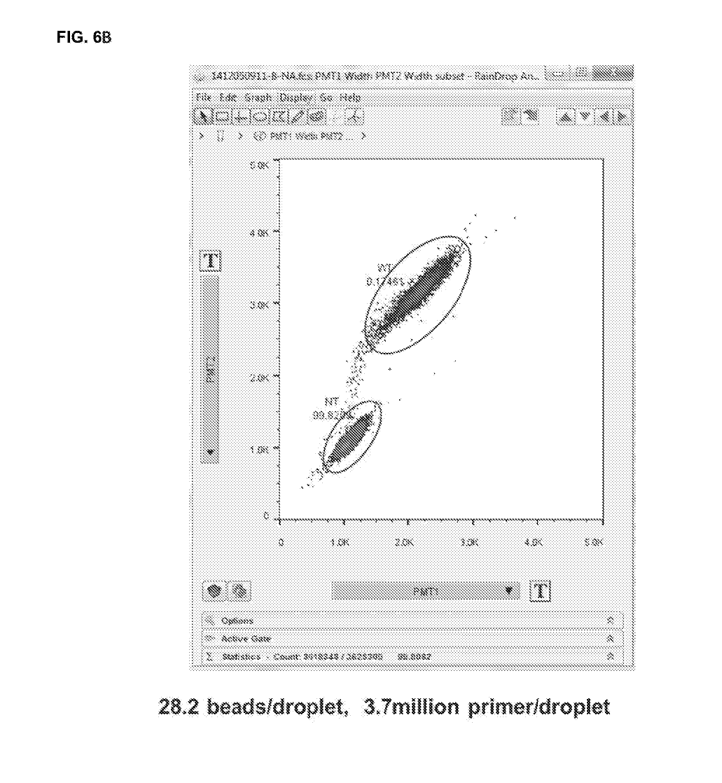

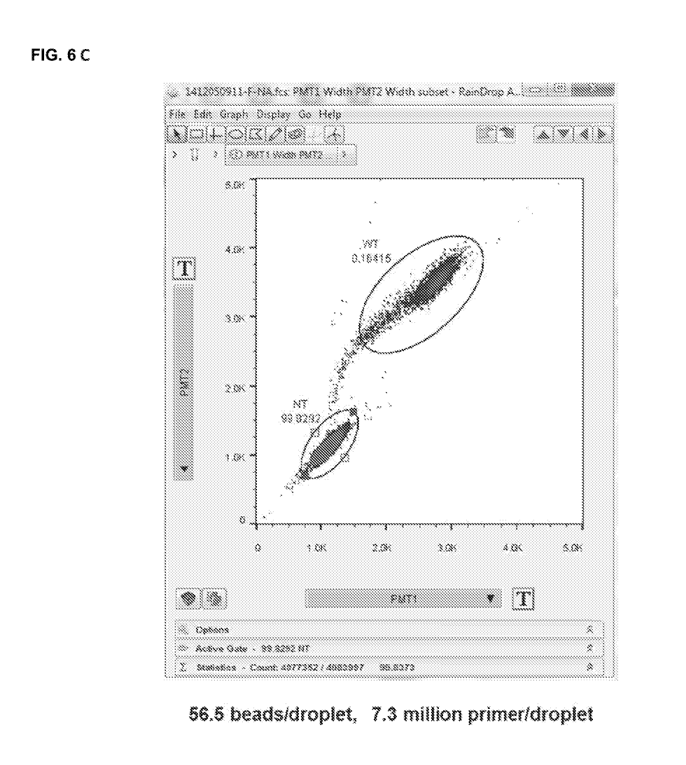

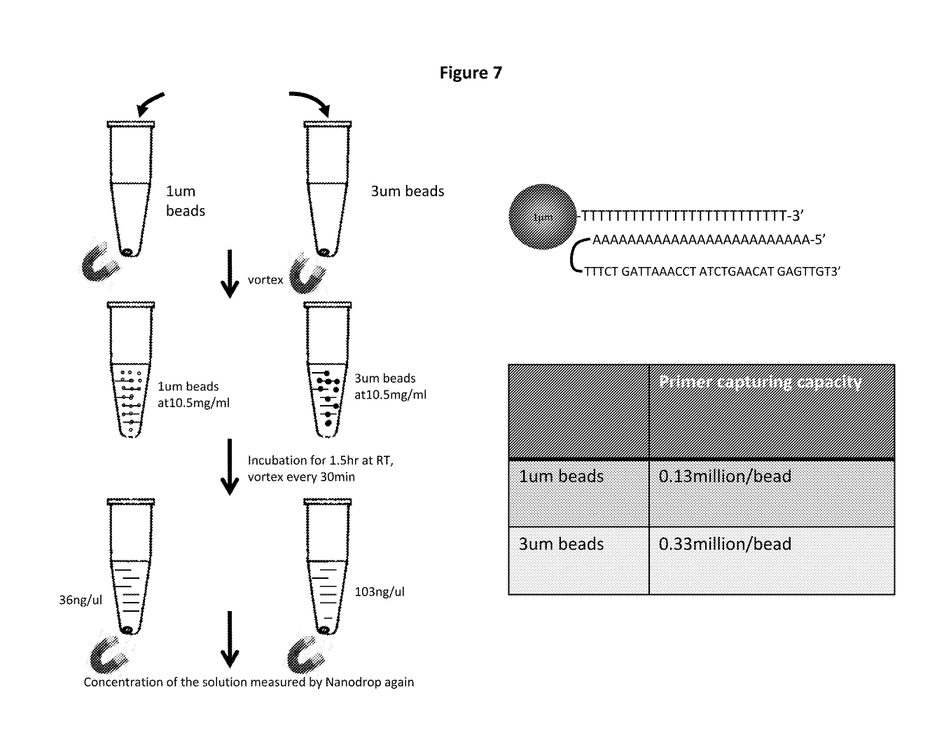

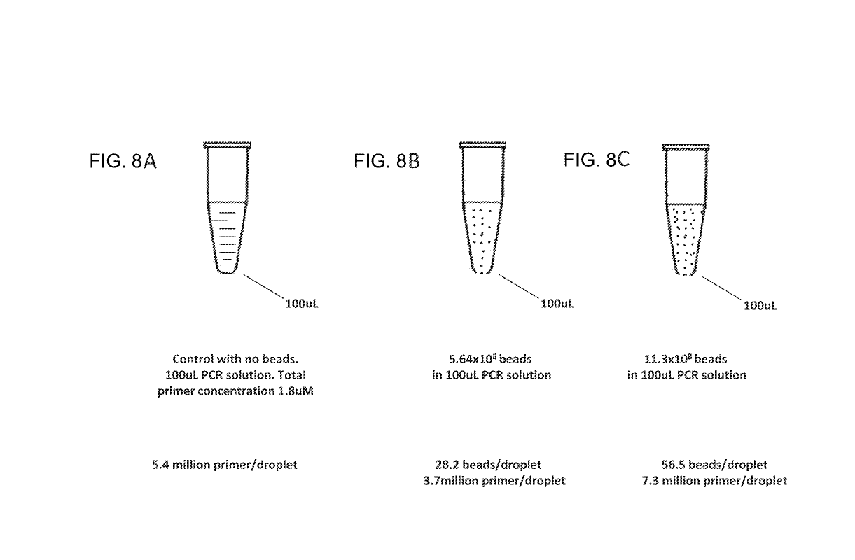

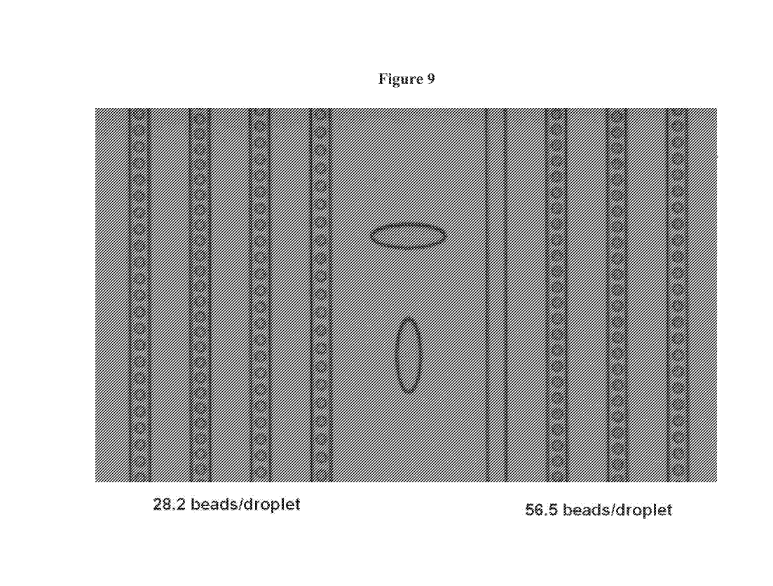

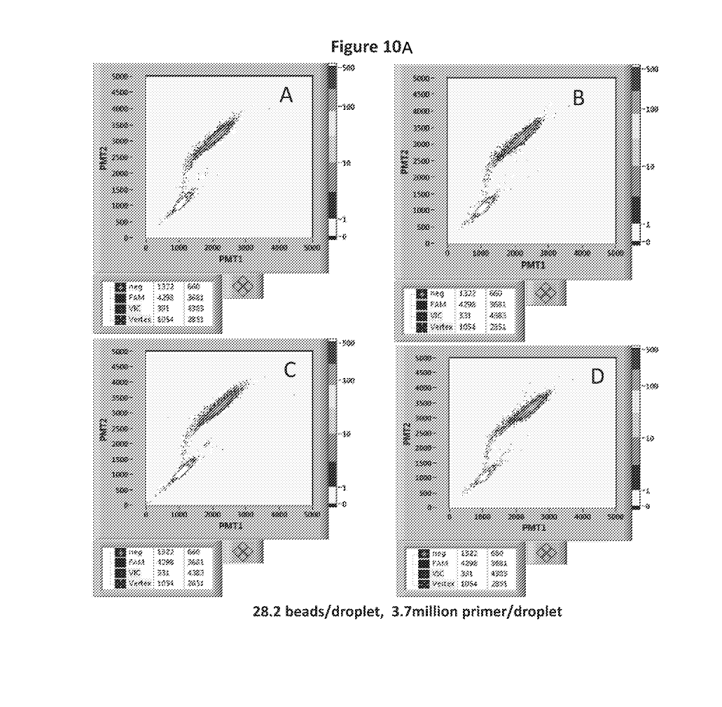

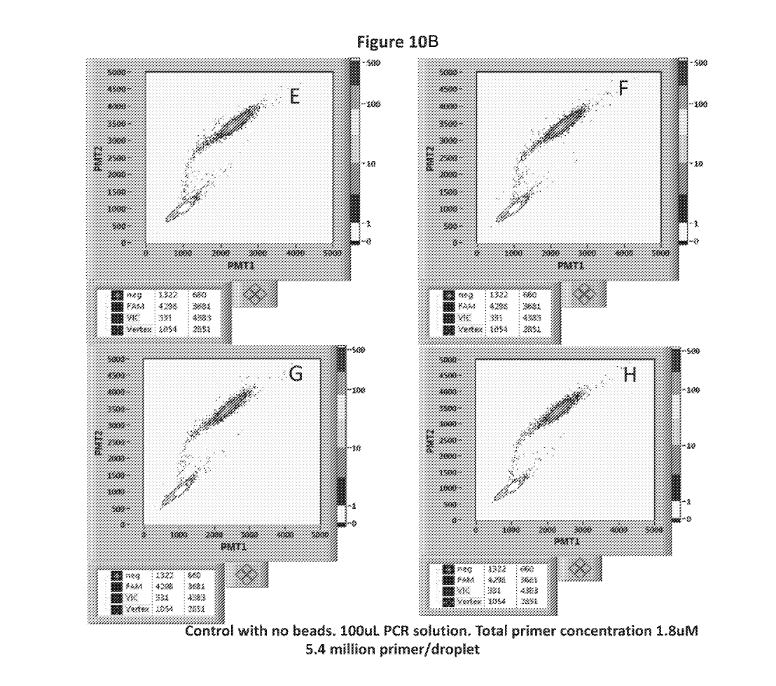

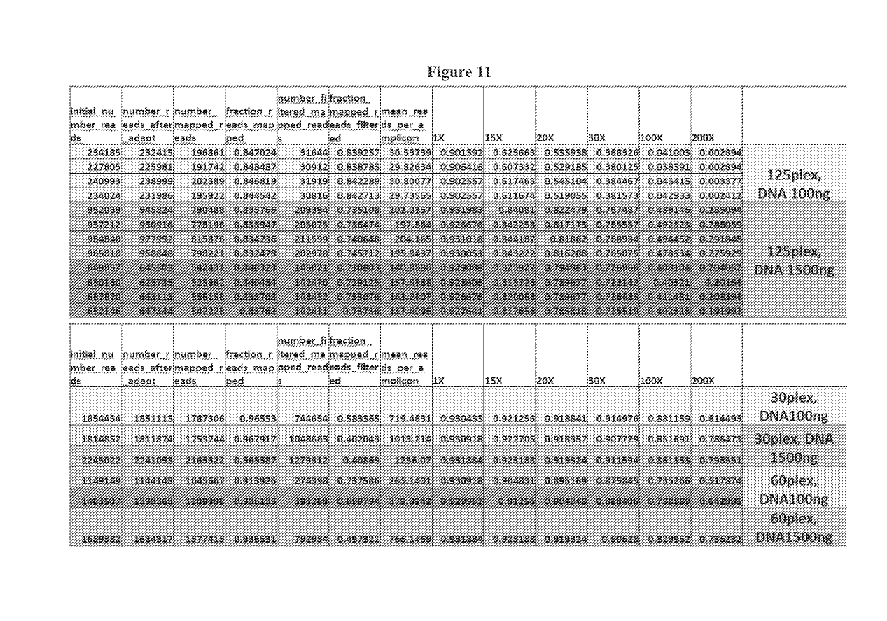

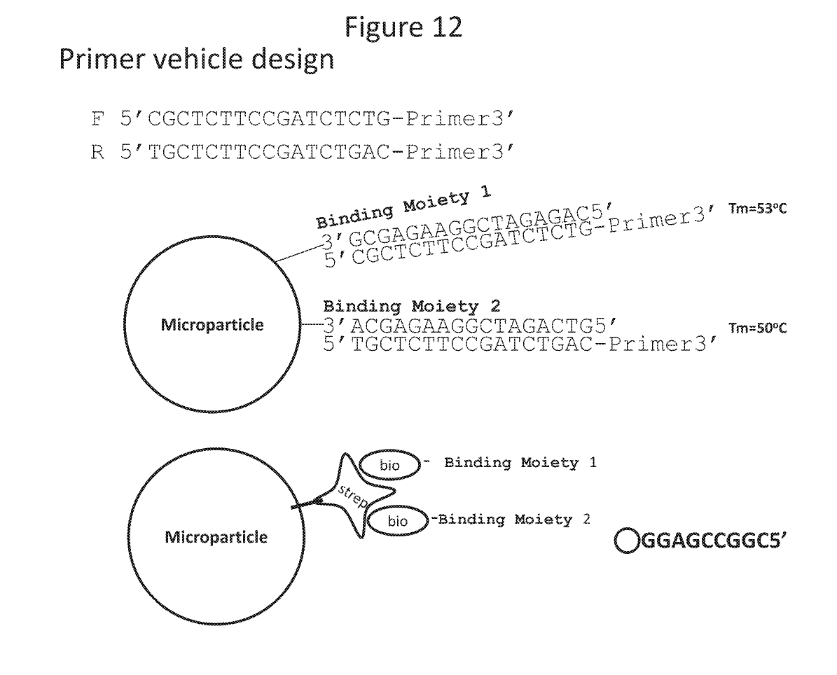

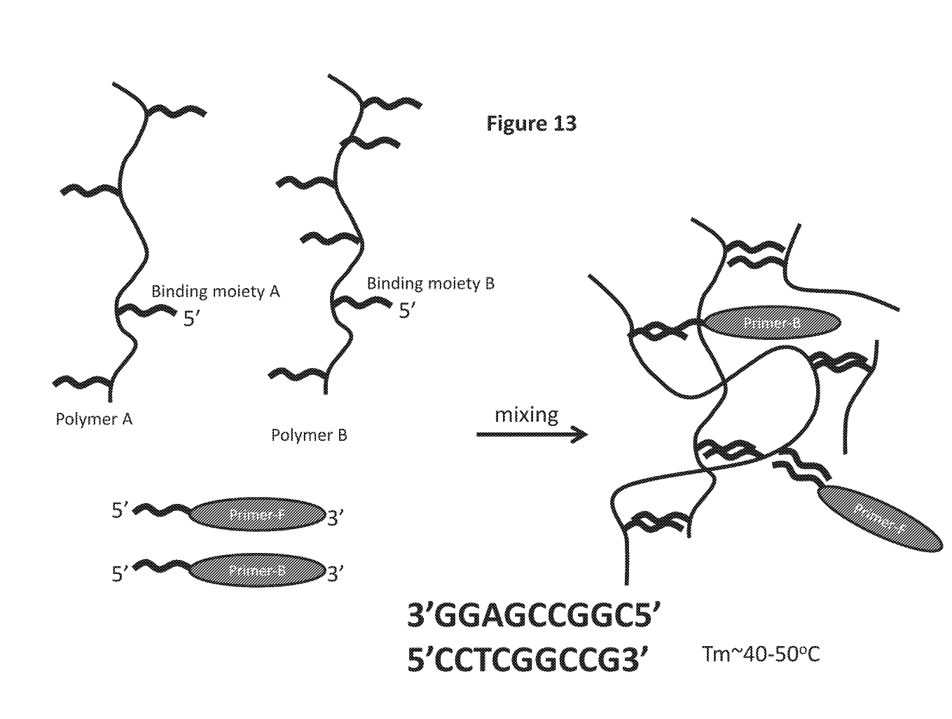

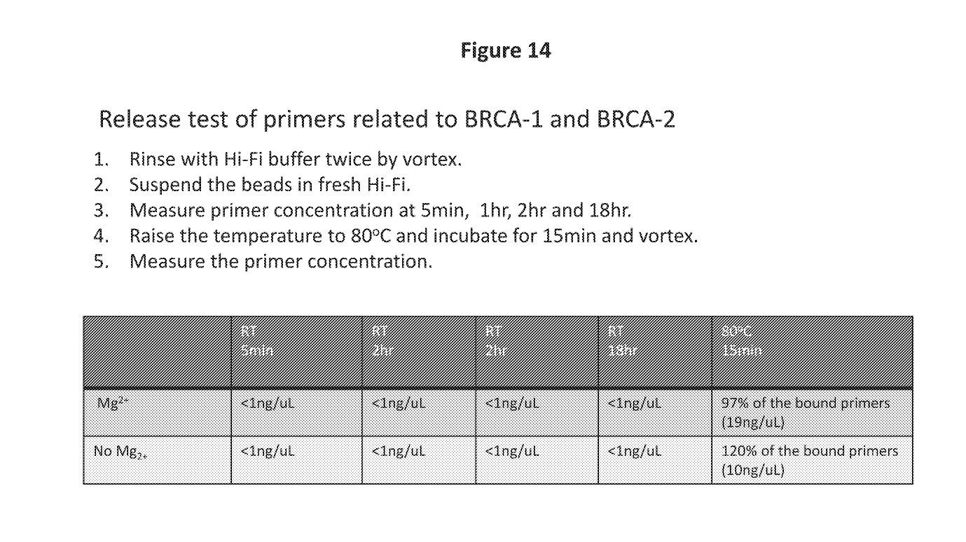

Yet another embodiment of bead 305 may include a Poly(DMAA-co-MAPPA)-Oligo that is a water soluble polymer with side chains of acetylene groups, reacted with azide group of azide-functionalized oligoDNA using what is sometimes referred to as “click chemistry” that is catalyzed by an application of Cu(I) (such as CuBr). An example of the reaction is illustrated in FIG. 5, the result is a water soluble polymer which can bind primer species to make thermal sensitive hydrogels with an Upper Critical Solution Temperature (sometimes referred to as “UCST”) transition property via hybridization interaction between complimentary nucleic acid.

As generally defined herein, R1 is optionally substituted alkylene, optionally substituted heteroalkylene, optionally substituted alkenylene, optionally substituted heteroalkenylene, optionally substituted alkynylene, optionally substituted heteroalkynylene, optionally substituted heterocyclylene, or optionally substituted heteroarylene. In certain embodiments, R1 is optionally substituted alkylene. In certain embodiments, R1 is substituted alkylene. In certain embodiments, R1 is unsubstituted alkylene. In certain embodiments, R1 is straight chain unsubstituted alkylene. In certain embodiments, R1 is optionally substituted C1-C8 alkylene.

As generally defined herein, R2 is hydrogen, substituted or unsubstituted alkyl, or a nitrogen protecting group. In certain embodiments, R2 is hydrogen. In certain embodiments, R2 is substituted or unsubstituted alkyl. In certain embodiments, R2 is a nitrogen protecting group.

As generally defined herein, R3 is hydrogen, substituted or unsubstituted alkyl, or a nitrogen protecting group. In certain embodiments, R3 is hydrogen. In certain embodiments, R3 is substituted or unsubstituted alkyl. In certain embodiments, R3 is a nitrogen protecting group.

As generally defined herein, R4 is optionally substituted alkylene, optionally substituted alkenylene, optionally substituted alkynylene, optionally substituted heterocyclylene, or optionally substituted heteroarylene. In certain embodiments, R4 is optionally substituted alkylene. In certain embodiments, R4 is substituted alkylene. In certain embodiments, R4 is unsubstituted alkylene. In certain embodiments, R4 is straight chain unsubstituted alkylene. In certain embodiments, R4 is optionally substituted C1-C8 alkylene.

In the present example, with methanol as solvent, much lower molecular weight product results and no gelation process occurs no matter how long the reaction time is. Therefore, a mixture of DMSO and methanol as solvent may be employed to obtain a high molecular weight product without risk of gelation. By doing so, a long reaction time can also be adopted to obtain a high conversion/yield.

### EXAMPLES

These and other aspects of the present invention will be further appreciated upon consideration of the following Examples, which are intended to illustrate certain particular embodiments of the invention but are not intended to limit its scope, as defined by the claims.

U.S. Patent Nos. 8765485, 7129091, 7655470, 7718578, 7901939, 8273573, 8304193, 9448172, 9029083, 9074242 , 9273308, 9328344, 9150852, 9399797, 9266104, 9029083, 8528589, 9012390, 9150852, 9176031, 8841071, 8658430, 7708949, 8337778, 8986628, and U.S. Patent Application Publication Nos. 2010-0022414, 2012-0264646, 2013-0260447, 2014-0295421, 2014-0045712, 2013-0260447, 2013-0295567, 2013-0295568, 2007-0092914, 2009-0005254, 2005/0221339, 2013-0109575, 2012-0122714, 2013-0064776, 2012-0244043, 2012-0219947, 2015-0126400, 2014-0113300, 2014-0303005, 2015-0167066, 2015-0099754, 2015-0184256, 2015-0283546, 2014-0305799, 2011-0190146 are all incorporated herein by reference in their entireties.

### Synthesis of Precursor MAPPA

About 150 ml anhydrous dichloromethane, 10 ml PPA, and 20 ml trimethylamine are mixed together in a 250 ml receptacle and 15 ml methacryloyl chloride was added in a drop wise fashion. The solution formed a precipitation of salt over a 2 hr reaction time that was then filtered by 0.2 um PTFE membrane to remove the salt precipitate. The solution was then rinsed with 50 ml DI water three times, and the organic phase dried over anhydrous Na2SO4 over night, and then dried by rotavap. The remaining liquid was filtered to remove any salt and optionally vacuum distilled to further purify the final product.

### Synthesis of Poly(DMAA-co-MAPPA) as Illustrated in FIG. 5

0.9 ml of N,N-Dimethylacrylamide, 0.1 ml MAPPA and 20 mg AIBN were dissolved in 10 ml DMSO in a 25 ml Schlenk reaction tube. The solution was deoxygenated by “vacuum-purge with argon” process for 3 times. The temperature was raised to 70° C. and the reaction was carried out for 3 hours. Diethyl ether was used to precipitated polymer from DMSO. The use of DMSO as a solvent facilitates formation of very high molecular weight product. When polymerization time is longer than 3 hours there may be an abrupt increase in viscosity and formation of cross-linked gel and therefore, polymerization time should not extend past 3 hours where the solution exhibits a moderately increased viscosity. Product can be precipitated by diethyl ether, and by dissolving in THF and precipitation in diethyl ether for multiple times to remove DMSO.

### Preparation of Primer Vehicles

Commercial microbeads with functionalized surface were mixed with primer solutions to capture primers at low temperature (<70° C.). The primer-loaded microbeads were mixed with PCR solution which was then divided into droplets on a microfludic device. Upon temperature increase (>70° C.), the primers were released from the bead surface into the solution phase.

Oligo d(T)₂₅ Magnetic Beads with diameters of 1 um or 3 um were used (obtained from New England Biolabs). The beads have Poly(dT)₂₅ attached on the surface at 5′ end of the poly(T)₂₅. The primers were designed for the SMN c88G assays. Poly(A)₂₅ was introduced at 5′ end for both forward and reverse primers to allow binding with the poly(T)₂₅ on the bead surface.

To measure the binding capacity of the beads for the primer, UV absorption at 280 nm of primer solution was measured before and after mixing with the beads at room temperature for 1.5 hr. Binding capacity was determined as 0.13 million primer/bead for 1 um bead and 0.33 million primer for 3 um bead, respectively. The primers captured on the beads also stayed stable on the beads at room temperature.

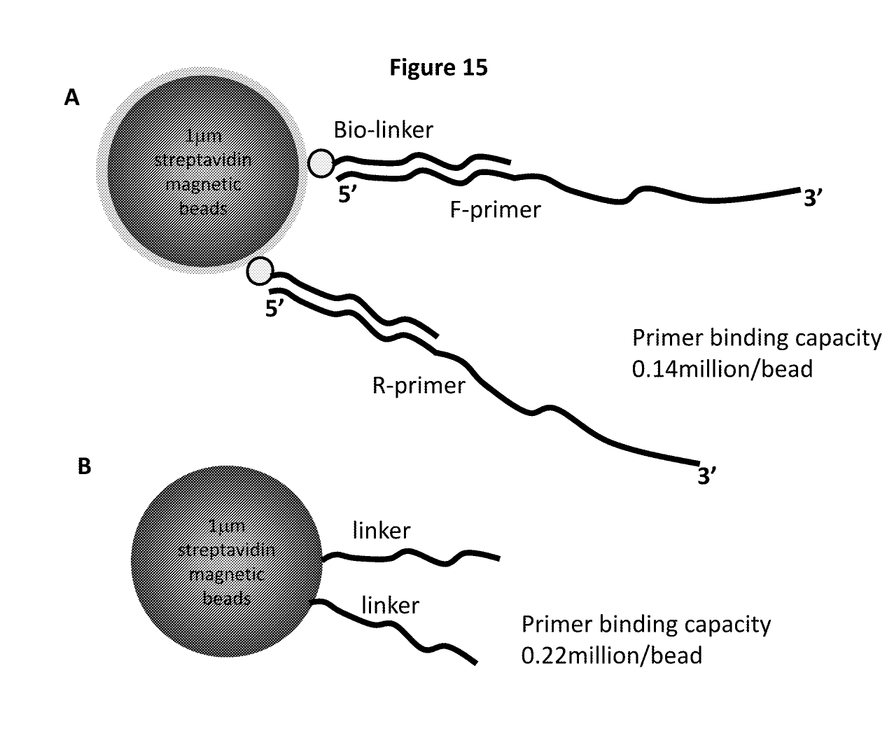

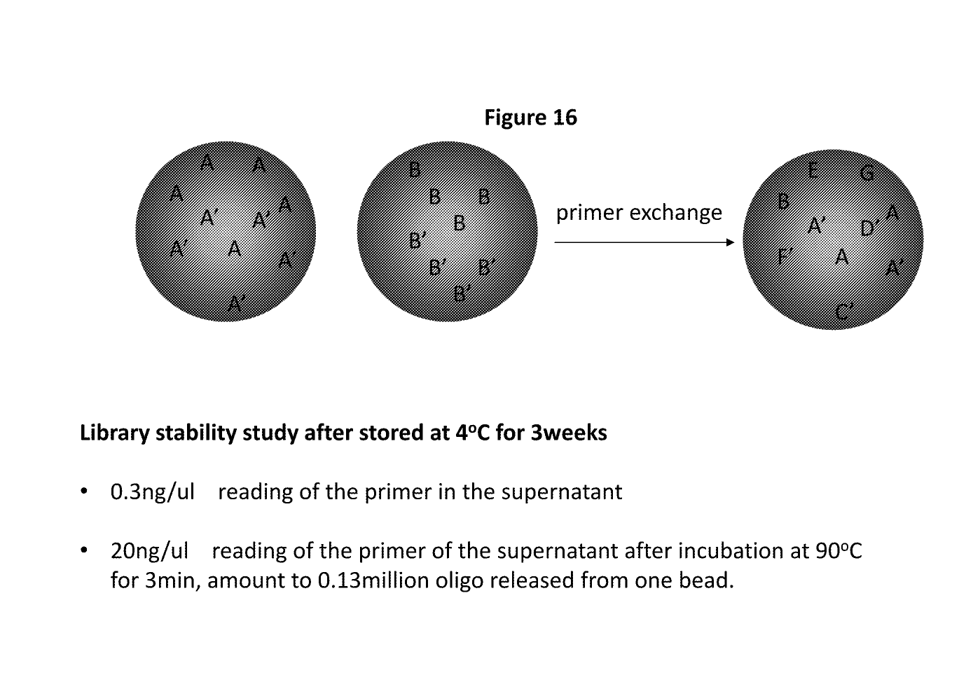

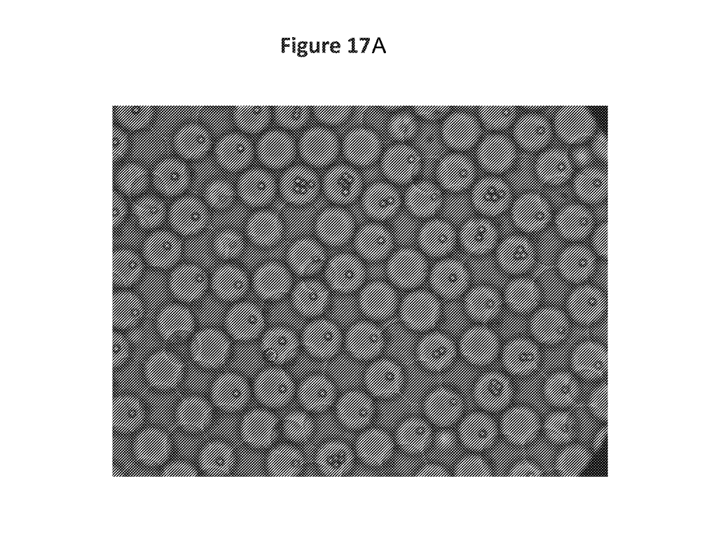

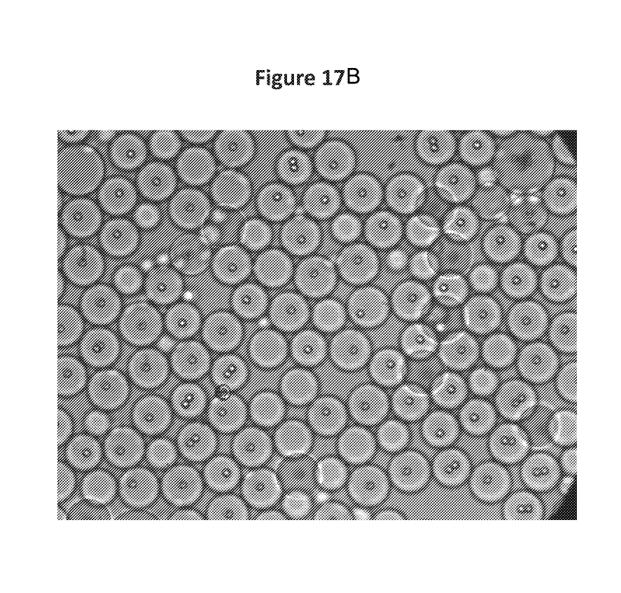

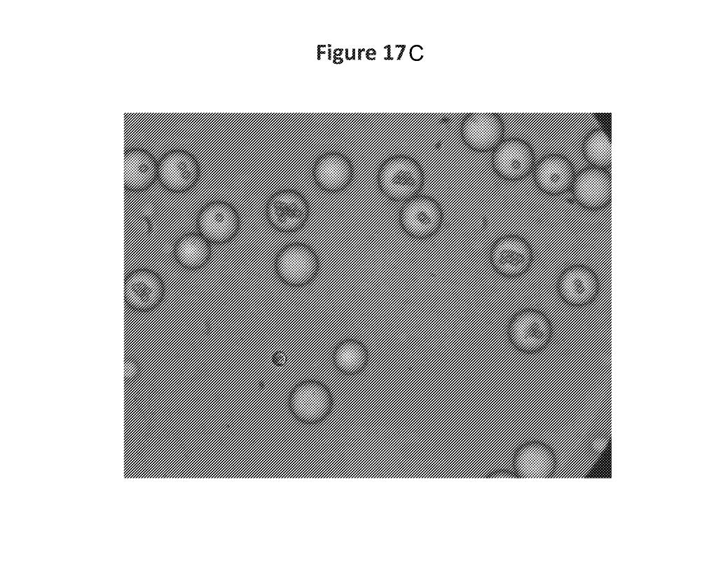

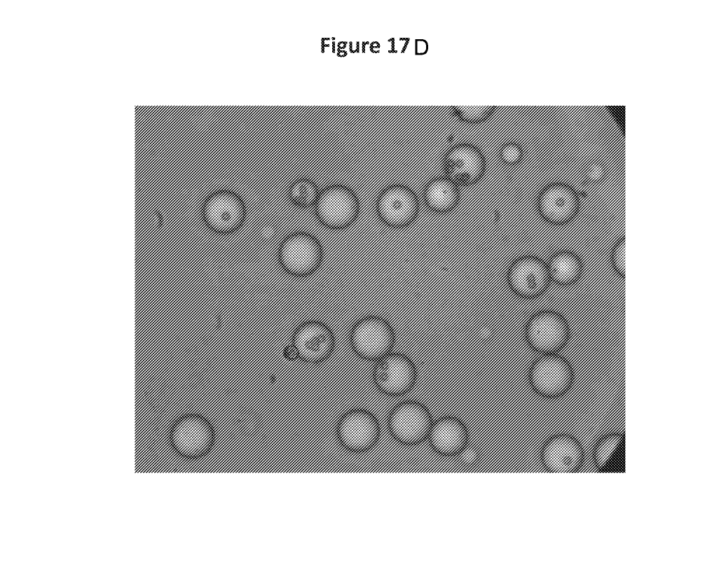

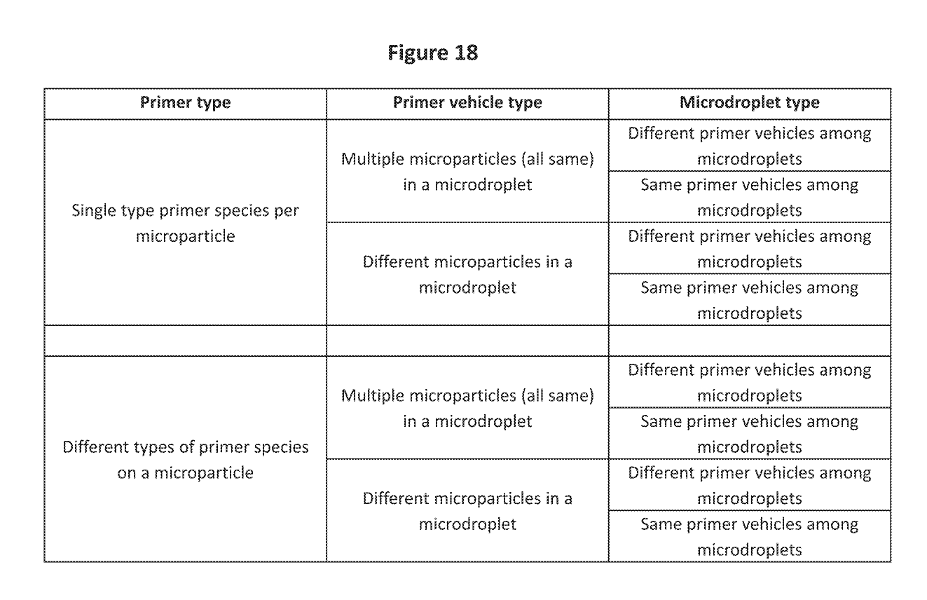

Two targets panels were used in making primer vehicle library. One panel contains 122 primer pairs, another one contain 2020 primer pairs. All the primer pairs in these two panels have the same sequence at 5′ as shown in FIG. 15. The capture capacity of beads was measured by UV absorption as 0.13 million oligos per beads, i.e. 0.065 million primer pairs per bead.

For the 122 panel library production, 10 ul of every primer pair solution at 4 uM in Tris buffer was added into vials in two 96-well plate. To every vial, 10 uL of beads suspension at 4 mg/ml in 2× Hi-Fi (Life Tech) was added. The plate was loaded into PCR thermal cycler with an annealing program which anneals the plate from 80° C. to 10° C. over one hour. The beads from different vials were collected and mixed, rinsed with Hi-Fi buffer three times to remove free primer oligos, suspended into Hi-Fi buffer at 4 mg/ml for storage at 4° C. In this library, every bead has a single primer pair.

For the 2020 panel library production, the 2020 primer pair was divided into 405 vials of two 384 well plate, with every vial contain 5 different primer pair with total volume of 10 ul and total concentration of 10 uM. To every vial 10 uL of beads suspension at 4 mg/ml in 2× Hi-Fi (Life Tech) was added. The plate was loaded into PCR thermal cycler with an annealing program which anneals the plate from 80° C. to 10° C. over one hour. The beads from different vials were collected and mixed, rinsed with Hi-Fi buffer three times to remove free primer oligos, suspended into Hi-Fi buffer at 4 mg/ml for storage at 4° C. In this library, every bead has 5 primer pair.

### Amplification

In a 1^{st} PCR reaction, beads carrying primers were mixed with Taqman Genotype Master Mix (Thermal Fisher) and DNA template, prepared into emulsion for amplification of targets. For the 122 panel, 24 uL beads suspension was taken for every 40 uL PCR solution in a PCR vial to reach an average bead number of 25 per droplet (5 pL). The supernatant of the beads suspension was removed after the beads were settle down to the bottom by a magnet, after which 40 uL PCR solution (Taqman Genotype Master Mix) containing 50 ng sheared human genome DNA (3k bp) was added and mixed with the beads. This mixture was made into 5 pL droplets on RainDance RainDrop Source system. For the 2020 Panel, 24, 12 and 6 uL beads suspension was added into every 40 uL reaction solution, leading to average number of 25, 12 and 6 beads per droplet (5 pL). Since each bead has 5 different primer pairs, the average primer pair number per droplet was 125, 60 and 6, respectively. DNA loading level was controlled at 100 ng or 1500 ng per 40 uL PCR solution. The solutions were made into 50 pL droplets on RainDrop Source system. The emulsions were loaded into thermal cycler for PCR reaction. Control experiments with mixed primer pair solutions added without beads as carrier were done to be compared with the bead containing sample.

### Sequencing

After the 1^{st} PCR reaction, the emulsion was broke and beads were removed. The aqueous phase obtained from the 1^{st} PCR amplification was used as template for the 2^{nd} PCR reaction. The second reaction use Hi-Fi master mix (Lift Tech) to introduce Illumina sequencer adaptors and did not utilize droplets. Samples were sequenced on Illumina Miseq seqencer.

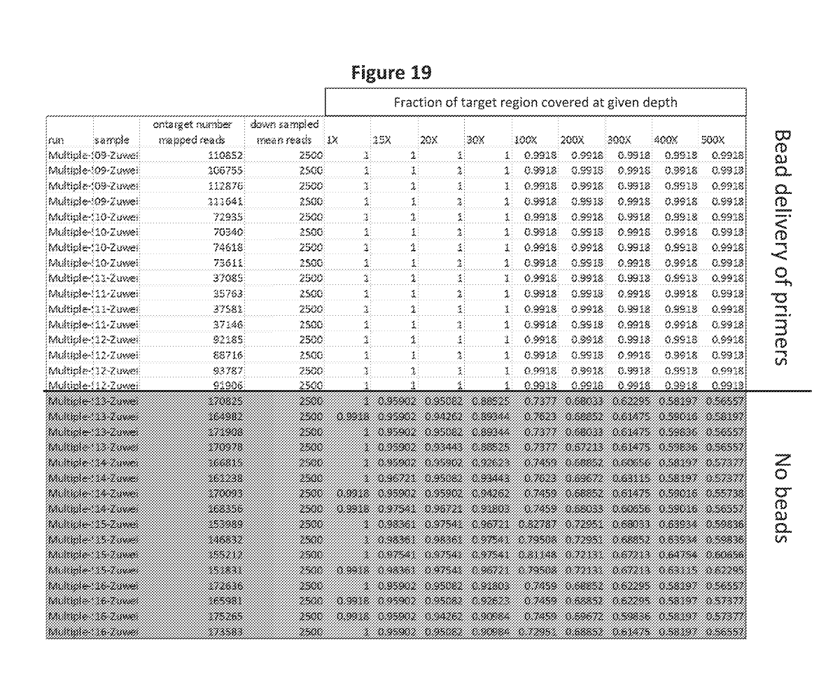

FIG. 19 and FIG. 11 show the sequencing results of the 122 primer panel and the 2020 primer panel. Both panels contain a large number of overlapping amplicons to tile across contiguous regions of the genome. These overlapping amplicons are typically extremely challenging to amplify in the same reaction as they tend to generate products predominantly consisting of the overlapping regions. The percentage of targets that were covered with mapping reads of more than 1, 15, 30, 100, 200, 300, 400, and 500 times were shown in the table of FIG. 19. For easy of comparison, the reads have been normalize to the same average depth of coverage of 2500 for each condition. FIG. 19 shows the coverage of the sample with beads was more uniform than the control samples without beads as can be seen by the significantly larger fraction of the target region that is covered at 500×(>99% in comparison to 55% to 60% when primer vehicle was not used, no beads). In FIG. 11, the control sample in which all 2020 primer pairs were mixed together in the 1^{st} PCR reaction showed no sequence mapping at all (date not shown), indicating no useful target amplification. With bead delivered primer species, samples have shown satisfactory mapping number for more than 90 percent targets, proving the concept that the random distribution of primer species over the droplets reduces the primer-primer interaction and target overlapping problems and improves uniformity of the amplification products.

Having described various embodiments and implementations, it should be apparent to those skilled in the relevant art that the foregoing is illustrative only and not limiting, having been presented by way of example only. Many other schemes for distributing functions among the various functional elements of the illustrated embodiments are possible. The functions of any element may be carried out in various ways in alternative embodiments.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A microparticle of Formula (I): 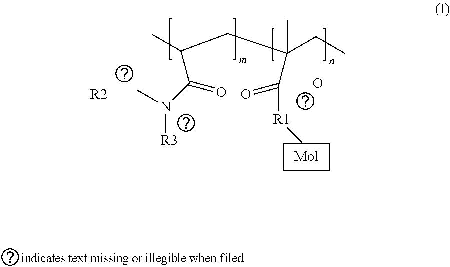
wherein [symbol]
is a biological molecule;
m is an integer of 0 to 100, inclusive;
n is an integer of 0 to 100, inclusive;
R1 is a linker selected from the group consisting of a bond, optionally substituted alkylene, optionally substituted heteroalkylene, optionally substituted alkenylene, optionally substituted heteroalkenylene, optionally substituted alkynylene, optionally substituted heteroalkynylene, optionally substituted heterocyclylene, or optionally substituted heteroarylene, and
each of R2 and R3 is independently hydrogen, substituted or unsubstituted alkyl, or a nitrogen protecting group.

2. The microparticle of claim 1, wherein the microparticle is of Formula (II): 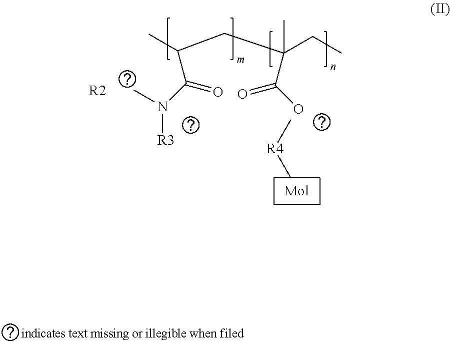
wherein R4 is optionally substituted alkylene, optionally substituted alkenylene, optionally substituted alkynylene, optionally substituted heterocyclylene, or optionally substituted heteroarylene.

3. The microparticle of claim 2, wherein the microparticle is of Formula (II-a): 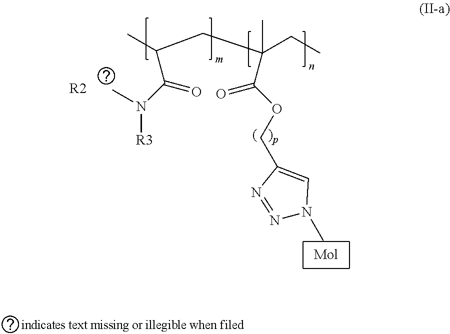
wherein p is an integer of 1 to 5, inclusive.

4. The microparticle of claim 1, wherein the microparticle is of Formula (II-b):

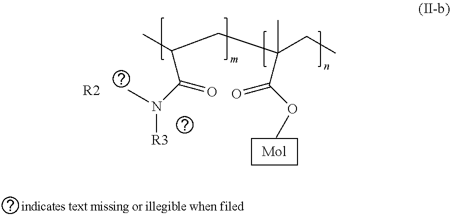

5-35. (canceled)

36. A plurality of microdroplets, each comprising a nucleic acid template molecule and a plurality of primer vehicles, wherein each primer vehicle comprises a plurality of primer species bound to a microparticle through a plurality of binding moieties, wherein each primer species is specific for a different target site of a nucleic acid template molecule.

37-38. (canceled)

39. The plurality of microdroplets of claim 36, wherein at least one primer species is specific for a target site on the nucleic acid template molecule in at least one microdroplet.

40-43 (canceled)

44. The plurality of microdroplets of claim 36, wherein each of the binding moieties comprises a sequence complementary to a member of the primer pairs.

45-50. (canceled)

51. The plurality of microdroplets of claim 36, wherein each of the primer vehicles has a single primer species bound.

52. (canceled)

53. The plurality of microdroplets according to any one of the preceding claims of claim 36, wherein each of the primer vehicles has different primer species bound.

54-55. (canceled)

56. The plurality of microdroplets claim 36, wherein each microdroplet has a plurality of same primer vehicles.

57-58. (canceled)

59. The plurality of microdroplets of claim 36, wherein each primer species is a primer pair.

60-64. (canceled)

65. The plurality of microdroplets of claim 36, wherein the primer species comprises a barcode, a universal sequence, and a target specific sequence.

66-71. (canceled)

72. A method for detecting a nucleic acid template molecule in a biological sample, comprising the steps of:
forming a plurality of microdroplets of any one of the preceding claims;
amplifying at least one nucleic acid template molecule in the microdroplets to give an amplified product; and
sequencing the amplified product.

73. The method of claim 72, wherein said forming step further comprises:
providing a first solution comprising a nucleic acid template molecule;
providing a second solution comprising a plurality of different primer species each specific for a different target site on a nucleic acid template;
merging the first and second solution to form a merged solution; and
partitioning the merged solution in an immiscible fluid.

74. The method of claim 72, before the sequencing step, further comprising merging one of the microdroplets with a microdroplet comprising a barcode.

75-78. (canceled)
